SEQUENCE LISTING (136 sequences)

SEQ ID NO: 1 - protein, 29 aa, synthetic construct
  REGION 1..29: note = epitope
VKFRKGSPENVEFKSGPGTEMALGAKPSA

SEQ ID NO: 2 - protein, 7 aa, synthetic construct
  REGION 1..7: note = epitope
KGSPENV

SEQ ID NO: 3 - protein, 105 aa, synthetic construct
  REGION 1..105: note = synthetic sequence
QIVLTQSPALMSASPGEKVTMTCSASSSISSMYWYQQKPRSSPKPWIYLTSNLASGVPARFSGSGSGTSYSLTISSMEAEDAATYYCQQWSSNLTFGAGTKLELK

SEQ ID NO: 4 - protein, 119 aa, synthetic construct
  REGION 1..119: note = 2H9chim_VH wt_aa
DVQLVESGGGLVQPGGSRKLSCAASGFTFSSFGMHWVRQAPEKGLEWVAYISSGSTTIYYADTVKGRFTISRDNPKNTLFLQMTSLRSEDTAMYFCARWTVVTNCFDYWGQGTTLTVSS

SEQ ID NO: 5 - protein, 107 aa, synthetic construct
  REGION 1..107: note = 3H8chim_VL wt_aa
DIVMTQSHKFMSTSVGDRVNITCKASQDVSTAVAWYQLKPGQSPKLLIYWASIRHTGVPDRFTGSGSGTDYTLTISSVQAEHLALYYCQQHYSTPWTFGGGTKLEIK

SEQ ID NO: 6 - protein, 120 aa, synthetic construct
  REGION 1..120: note = 3H8chim_VH wt_aa
QIQLVQSGPELKKPGETVNISCMASGYSFTNYGMNWVKQAPGKGLKWMGWINTYTGEPTYADDFKGRFAFSLETSASTAYLQINNLKNEDMATYFCARQPYDYVPSFPYWGQGTLVTVSA

SEQ ID NO: 7 - protein, 112 aa, synthetic construct
  REGION 1..112: note = 26F10chim_VL wt_aa
DVVMPQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPLTFGAGTKLELK

SEQ ID NO: 8 - protein, 119 aa, synthetic construct
  REGION 1..119: note = 26F10chim_VH wt_aa
EVQLQQSGTELVRSGASVKLSCTTSGFNIKDYYMHWVKQRPEQGLEWIGWIDPENGDTEYAPKFQGKATMTADTSSNTAYLQLSSLTSEDTAVYYCNAYDGYYGAMDYWGQGTSVTVSS

SEQ ID NO: 9 - protein, 111 aa, synthetic construct
  REGION 1..111: note = 13F7chim_VL wt_aa
NIVLTQSPASLAVSLGQRATISCRASESVDSYGNSFMHWYQQKPGQPPKLLIYRASNLESGIPARFSGSGSRTDFTLTINPVEADDVATYYCQQSNEDPPTFGGGTKLEIK

SEQ ID NO: 10 - protein, 119 aa, synthetic construct
  REGION 1..119: note = 13F7chim_VH wt_aa
QVQLQQSGAELVRPGVSVKVSCKGSGYTFTDYAMHWVKQSHPKSLEWIGVIGTYYGDAYYNQNFKGKATMTVDKSSSTAYMELARLTSEDSAIYYCARRAGTSDWFAYWGQGTLVTVSA

SEQ ID NO: 11 - protein, 107 aa, synthetic construct
  REGION 1..107: note = 11B5chim_VL wt_aa
DIQMTQSPASLSASVGETVTITCRASENIYSYLAWYQQKQGKSPQLLVYNGITLAEGVPSRFSGSGSGTQFSLKINSLQPEDFGDYYCQHHYGSPRTFGGGTTLEIK

SEQ ID NO: 12 - protein, 120 aa, synthetic construct
  REGION 1..120: note = 11B5chim_VH wt_aa
EVQLVESGGDLVKPGGSLKLSCAASGFAFSDYYMYWVRQTPEKRLEWVATISDDGIYTYYPDSVKGRFTISRDNAKNNLFLQMSSLKSEDTAMYYCARGGYRDYYAMDYWGQGTSVTVSS

SEQ ID NO: 13 - protein, 212 aa, synthetic construct
  REGION 1..212: note = full length 2H9chim_VL wt_aa
QIVLTQSPALMSASPGEKVTMTCSASSSISSMYWYQQKPRSSPKPWIYLTSNLASGVPARFSGSGSGTSYSLTISSMEAEDAATYYCQQWSSNLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 446 aa, synthetic construct
  REGION 1..446: note = full length 2H9chim_VH wt_aa
DVQLVESGGGLVQPGGSRKLSCAASGFTFSSFGMHWVRQAPEKGLEWVAYISSGSTTIYYADTVKGRFTISRDNPKNTLFLQMTSLRSEDTAMYFCARWTVVTNCFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 15 - protein, 214 aa, synthetic construct
  REGION 1..214: note = full length 3H8chim_VL wt_aa
DIVMTQSHKFMSTSVGDRVNITCKASQDVSTAVAWYQLKPGQSPKLLIYWASIRHTGVPDRFTGSGSGTDYTLTISSVQAEHLALYYCQQHYSTPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 447 aa, synthetic construct
  REGION 1..447: note = full length 3H8chim_VH wt_aa
QIQLVQSGPELKKPGETVNISCMASGYSFTNYGMNWVKQAPGKGLKWMGWINTYTGEPTYADDFKGRFAFSLETSASTAYLQINNLKNEDMATYFCARQPYDYVPSFPYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 17 - protein, 219 aa, synthetic construct
  REGION 1..219: note = full length 26F10chim_VL wt_aa
DVVMPQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 446 aa, synthetic construct
  REGION 1..446: note = full length 26F10chim_VH wt_aa
EVQLQQSGTELVRSGASVKLSCTTSGFNIKDYYMHWVKQRPEQGLEWIGWIDPENGDTEYAPKFQGKATMTADTSSNTAYLQLSSLTSEDTAVYYCNAYDGYYGAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 19 - protein, 218 aa, synthetic construct
  REGION 1..218: note = full length 13F7chim_VL wt_aa
NIVLTQSPASLAVSLGQRATISCRASESVDSYGNSFMHWYQQKPGQPPKLLIYRASNLESGIPARFSGSGSRTDFTLTINPVEADDVATYYCQQSNEDPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 446 aa, synthetic construct
  REGION 1..446: note = full length 13F7chim_VH wt_aa
QVQLQQSGAELVRPGVSVKVSCKGSGYTFTDYAMHWVKQSHPKSLEWIGVIGTYYGDAYYNQNFKGKATMTVDKSSSTAYMELARLTSEDSAIYYCARRAGTSDWFAYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 21 - protein, 214 aa, synthetic construct
  REGION 1..214: note = full length 11B5chim_VL wt_aa
DIQMTQSPASLSASVGETVTITCRASENIYSYLAWYQQKQGKSPQLLVYNGITLAEGVPSRFSGSGSGTQFSLKINSLQPEDFGDYYCQHHYGSPRTFGGGTTLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 447 aa, synthetic construct
  REGION 1..447: note = full length 11B5chim_VH wt_aa
EVQLVESGGDLVKPGGSLKLSCAASGFAFSDYYMYWVRQTPEKRLEWVATISDDGIYTYYPDSVKGRFTISRDNAKNNLFLQMSSLKSEDTAMYYCARGGYRDYYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 23 - protein, 5 aa, synthetic construct
  REGION 1..5: note = 2H9chim_VL wt_CDR1_aa
SSISS

SEQ ID NO: 24 - skipped

SEQ ID NO: 25 - protein, 8 aa, synthetic construct
  REGION 1..8: note = 2H9chim_VL wt_CDR3_aa
QQWSSNLT

SEQ ID NO: 26 - protein, 8 aa, synthetic construct
  REGION 1..8: note = 2H9chim_VH wt_CDR1_aa
GFTFSSFG

SEQ ID NO: 27 - protein, 8 aa, synthetic construct
  REGION 1..8: note = 2H9chim_VH wt_CDR2_aa
ISSGSTTI

SEQ ID NO: 28 - protein, 12 aa, synthetic construct
  REGION 1..12: note = 2H9chim_VH wt_CDR3_aa
ARWTVVTNCFDY

SEQ ID NO: 29 - protein, 6 aa, synthetic construct
  REGION 1..6: note = 3H8chim_VL wt_CDR1_aa
QDVSTA

SEQ ID NO: 30 - skipped

SEQ ID NO: 31 - protein, 9 aa, synthetic construct
  REGION 1..9: note = 3H8chim_VL wt_CDR3_aa
QQHYSTPWT

SEQ ID NO: 32 - protein, 8 aa, synthetic construct
  REGION 1..8: note = 3H8chim_VH wt_CDR1_aa
GYSFTNYG

SEQ ID NO: 33 - protein, 8 aa, synthetic construct
  REGION 1..8: note = 3H8chim_VH wt_CDR2_aa
INTYTGEP

SEQ ID NO: 34 - protein, 13 aa, synthetic construct
  REGION 1..13: note = 3H8chim_VH wt_CDR3_aa
ARQPYDYVPSFPY

SEQ ID NO: 35 - protein, 12 aa, synthetic construct
  REGION 1..12: note = 26F10chim_VL wt_CDR1_aa
QSLVHSNGNTYL

SEQ ID NO: 36 - skipped

SEQ ID NO: 37 - protein, 9 aa, synthetic construct
  REGION 1..9: note = 26F10chim_VL wt_CDR3_aa
SQNTHVPLT

SEQ ID NO: 38 - protein, 8 aa, synthetic construct
  REGION 1..8: note = 26F10chim_VH wt_CDR1_aa
GFNIKDYY

SEQ ID NO: 39 - protein, 8 aa, synthetic construct
  REGION 1..8: note = 26F10chim_VH wt_CDR2_aa
IDPENGDT

SEQ ID NO: 40 - protein, 12 aa, synthetic construct
  REGION 1..12: note = 26F10chim_VH wt_CDR3_aa
NAYDGYYGAMDY

SEQ ID NO: 41 - protein, 10 aa, synthetic construct
  REGION 1..10: note = 13F7chim_VL wt_CDR1_aa
ESVDSYGNSF

SEQ ID NO: 42 - skipped

SEQ ID NO: 43 - protein, 10 aa, synthetic construct
  REGION 1..10: note = 13F7chim_VL wt_CDR3_aa
QQSNEDPPTF

SEQ ID NO: 44 - protein, 8 aa, synthetic construct
  REGION 1..8: note = 13F7chim_VH wt_CDR1_aa
GYTFTDYA

SEQ ID NO: 45 - protein, 8 aa, synthetic construct
  REGION 1..8: note = 13F7chim_VH wt_CDR2_aa
IGTYYGDA

SEQ ID NO: 46 - protein, 13 aa, synthetic construct
  REGION 1..13: note = 13F7chim_VH wt_CDR3_aa
CARRAGTSDWFAY

SEQ ID NO: 47 - protein, 6 aa, synthetic construct
  REGION 1..6: note = 11B5chim_VL wt_CDR1_aa
ENIYSY

SEQ ID NO: 48 - skipped

SEQ ID NO: 49 - protein, 9 aa, synthetic construct
  REGION 1..9: note = 11B5chim_VL wt_CDR3_aa
QHHYGSPRT

SEQ ID NO: 50 - protein, 8 aa, synthetic construct
  REGION 1..8: note = 11B5chim_VH wt_CDR1_aa
GFAFSDYY

SEQ ID NO: 51 - protein, 8 aa, synthetic construct
  REGION 1..8: note = 11B5chim_VH wt_CDR2_aa
ISDDGIYT

SEQ ID NO: 52 - protein, 13 aa, synthetic construct
  REGION 1..13: note = 11B5chim_VH wt_CDR3_aa
ARGGYRDYYAMDY

SEQ ID NO: 53 - protein, 26 aa, synthetic construct
  REGION 1..26: note = 2H9chim_VL wt_FR1_aa
QIVLTQSPALMSASPGEKVTMTCSAS

SEQ ID NO: 54 - protein, 17 aa, synthetic construct
  REGION 1..17: note = 2H9chim_VL wt_FR2_aa
MYWYQQKPRSSPKPWIY

SEQ ID NO: 55 - protein, 36 aa, synthetic construct
  REGION 1..36: note = 2H9chim_VL wt_FR3_aa
NLASGVPARFSGSGSGTSYSLTISSMEAEDAATYYC

SEQ ID NO: 56 - protein, 10 aa, synthetic construct
  REGION 1..10: note = 2H9chim_VL wt_FR4_aa
FGAGTKLELK

SEQ ID NO: 57 - protein, 25 aa, synthetic construct
  REGION 1..25: note = 2H9chim_VH wt_FR1_aa
DVQLVESGGGLVQPGGSRKLSCAAS

SEQ ID NO: 58 - protein, 17 aa, synthetic construct
  REGION 1..17: note = 2H9chim_VH wt_FR2_aa
MHWVRQAPEKGLEWVAY

SEQ ID NO: 59 - protein, 38 aa, synthetic construct
  REGION 1..38: note = 2H9chim_VH wt_FR3_aa
YYADTVKGRFTISRDNPKNTLFLQMTSLRSEDTAMYFC

SEQ ID NO: 60 - protein, 11 aa, synthetic construct
  REGION 1..11: note = 2H9chim_VH wt_FR4_aa
WGQGTTLTVSS

SEQ ID NO: 61 - protein, 26 aa, synthetic construct
  REGION 1..26: note = 3H8chim_VL wt_FR1_aa
DIVMTQSHKFMSTSVGDRVNITCKAS

SEQ ID NO: 62 - protein, 17 aa, synthetic construct
  REGION 1..17: note = 3H8chim_VL wt_FR2_aa
VAWYQLKPGQSPKLLIY

SEQ ID NO: 63 - protein, 36 aa, synthetic construct
  REGION 1..36: note = 3H8chim_VL wt_FR3_aa
IRHTGVPDRFTGSGSGTDYTLTISSVQAEHLALYYC

SEQ ID NO: 64 - protein, 10 aa, synthetic construct
  REGION 1..10: note = 3H8chim_VL wt_FR4_aa
FGGGTKLEIK

SEQ ID NO: 65 - protein, 25 aa, synthetic construct
  REGION 1..25: note = 3H8chim_VH wt_FR1_aa
QIQLVQSGPELKKPGETVNISCMAS

SEQ ID NO: 66 - protein, 17 aa, synthetic construct
  REGION 1..17: note = 3H8chim_VH wt_FR2_aa
MNWVKQAPGKGLKWMGW

SEQ ID NO: 67 - protein, 38 aa, synthetic construct
  REGION 1..38: note = 3H8chim_VH wt_FR3_aa
TYADDFKGRFAFSLETSASTAYLQINNLKNEDMATYFC

SEQ ID NO: 68 - protein, 11 aa, synthetic construct
  REGION 1..11: note = 3H8chim_VH wt_FR4_aa
WGQGTLVTVSA

SEQ ID NO: 69 - protein, 26 aa, synthetic construct
  REGION 1..26: note = 26F10chim_VL wt_FR1_aa
DVVMPQTPLSLPVSLGDQASISCRSS

SEQ ID NO: 70 - protein, 16 aa, synthetic construct
  REGION 1..16: note = 26F10chim_VL wt_FR2_aa
HWYLQKPGQSPKLLIY

SEQ ID NO: 71 - protein, 36 aa, synthetic construct [sequence omitted]

SEQ ID NO: 72 - protein, 10 aa, synthetic construct
  REGION 1..10: note = 26F10chim_VL wt_FR4_aa
FGAGTKLELK

SEQ ID NO: 73 - protein, 25 aa, synthetic construct
  REGION 1..25: note = 26F10chim_VH wt_FR1_aa
EVQLQQSGTELVRSGASVKLSCTTS

SEQ ID NO: 74 - protein, 17 aa, synthetic construct
  REGION 1..17: note = 26F10chim_VH wt_FR2_aa
MHWVKQRPEQGLEWIGW

SEQ ID NO: 75 - protein, 38 aa, synthetic construct
  REGION 1..38: note = 26F10chim_VH wt_FR3_aa
EYAPKFQGKATMTADTSSNTAYLQLSSLTSEDTAVYYC

SEQ ID NO: 76 - protein, 11 aa, synthetic construct
  REGION 1..11: note = 26F10chim_VH wt_FR4_aa
WGQGTSVTVSS

SEQ ID NO: 77 - protein, 26 aa, synthetic construct
  REGION 1..26: note = 13F7chim_VL wt_FR1_aa
NIVLTQSPASLAVSLGQRATISCRAS

SEQ ID NO: 78 - protein, 17 aa, synthetic construct
  REGION 1..17: note = 13F7chim_VL wt_FR2_aa
MHWYQQKPGQPPKLLIY

SEQ ID NO: 79 - protein, 36 aa, synthetic construct
  REGION 1..36: note = 13F7chim_VL wt_FR3_aa
NLESGIPARFSGSGSRTDFTLTINPVEADDVATYYC

SEQ ID NO: 80 - protein, 9 aa, synthetic construct
  REGION 1..9: note = 13F7chim_VL wt_FR4_aa
GGGTKLEIK

SEQ ID NO: 81 - protein, 25 aa, synthetic construct
  REGION 1..25: note = 13F7chim_VH wt_FR1_aa
QVQLQQSGAELVRPGVSVKVSCKGS

SEQ ID NO: 82 - protein, 17 aa, synthetic construct
  REGION 1..17: note = 13F7chim_VH wt_FR2_aa
MHWVKQSHPKSLEWIGV

SEQ ID NO: 83 - protein, 37 aa, synthetic construct
  REGION 1..37: note = 13F7chim_VH wt_FR3_aa
YYNQNFKGKATMTVDKSSSTAYMELARLTSEDSAIYY

SEQ ID NO: 84 - protein, 11 aa, synthetic construct
  REGION 1..11: note = 13F7chim_VH wt_FR4_aa
WGQGTLVTVSA

SEQ ID NO: 85 - protein, 26 aa, synthetic construct
  REGION 1..26: note = 11B5chim_VL wt_FR1_aa
DIQMTQSPASLSASVGETVTITCRAS

SEQ ID NO: 86 - protein, 17 aa, synthetic construct
  REGION 1..17: note = 11B5chim_VL wt_FR2_aa
LAWYQQKQGKSPQLLVY

SEQ ID NO: 87 - protein, 36 aa, synthetic construct
  REGION 1..36: note = 11B5chim_VL wt_FR3_aa
TLAEGVPSRFSGSGSGTQFSLKINSLQPEDFGDYYC

SEQ ID NO: 88 - protein, 10 aa, synthetic construct
  REGION 1..10: note = 11B5chim_VL wt_FR4_aa
FGGGTTLEIK

SEQ ID NO: 89 - protein, 25 aa, synthetic construct
  REGION 1..25: note = 11B5chim_VH wt_FR1_aa
EVQLVESGGDLVKPGGSLKLSCAAS

SEQ ID NO: 90 - protein, 17 aa, synthetic construct
  REGION 1..17: note = 11B5chim_VH wt_FR2_aa
MYWVRQTPEKRLEWVAT

SEQ ID NO: 91 - protein, 38 aa, synthetic construct
  REGION 1..38: note = 11B5chim_VH wt_FR3_aa
YYPDSVKGRFTISRDNAKNNLFLQMSSLKSEDTAMYYC

SEQ ID NO: 92 - protein, 11 aa, synthetic construct
  REGION 1..11: note = 11B5chim_VH wt_FR4_aa
WGQGTSVTVSS

SEQ ID NO: 93 - DNA, 316 bp, synthetic construct
  misc_feature 1..316: note = 2H9chim_VL wt
  source 1..316: mol_type = other DNA
caaattgttctcacccagtctccagcactcatgtctgcatctccaggggagaaggtcaccatgacctgcagtgccagctcaagtataagttccatgtactggtaccagcagaagccaagatcctcccccaaaccctggatttatctcacatccaacctggcttctggagtccctgctcgcttcagtggcagtggatctgggacctcttactctctcacaatcagcagcatggaggctgaggatgctgccacctattactgccagcagtggagtagtaacctcacgttcggtgctgggaccaagctggagctgaaac

SEQ ID NO: 94 - DNA, 358 bp, synthetic construct
  misc_feature 1..358: note = 2H9chim_VH wt
  source 1..358: mol_type = other DNA
gatgtgcagctggtggagtctgggggaggcttagtgcagcctggagggtcccggaaactctcctgtgcagcctctggattcactttcagtagctttggaatgcactgggttcgtcaggctccagagaaggggctggagtgggtcgcatacattagtagtggcagtactaccatctactatgcagacacagtgaagggccgattcaccatctccagagacaatcccaagaacaccctgttcctgcaaatgaccagtctaaggtctgaggacacggccatgtatttctgtgcaagatggacggtagtaacaaactgctttgactactggggccaaggcaccactctcacagtctcctcag

SEQ ID NO: 95 - DNA, 322 bp, synthetic construct
  misc_feature 1..322: note = 3H8chim_VL wt
  source 1..322: mol_type = other DNA
gacattgtgatgacccagtctcacaaattcatgtccacatcagtaggagacagggtcaacatcacctgcaaggccagtcaggatgtgagtactgctgttgcctggtatcaactaaaaccagggcaatctcctaaactactgatttactgggcatctattcggcacactggagtccctgatcgcttcacaggcagtggatctgggacagattatactctcaccatcagcagtgtgcaggctgaacacctggcactttattactgtcagcaacattatagtactccgtggacgttcggtggaggcaccaagctggagatcaaac

SEQ ID NO: 96 - DNA, 361 bp, synthetic construct
  misc_feature 1..361: note = 3H8chim_VH wt
  source 1..361: mol_type = other DNA
cagatccagttggtgcagtctggacctgagctgaagaagcctggagaaacagtcaacatctcctgcatggcttctgggtattccttcacaaactatggaatgaactgggtgaagcaggctccaggaaagggtttaaagtggatgggctggataaacacctacactggagagccaacatatgctgatgacttcaagggacgctttgccttctctttggaaacctctgccagcactgcctatttgcagatcaacaacctcaaaaatgaggacatggctacatatttctgtgcaaggcagccgtatgattacgtcccctcgtttccttactggggccaagggactctggtcactgtctctgcag

SEQ ID NO: 97 - DNA, 337 bp, synthetic construct
  misc_feature 1..337: note = 26F10chim_VL wt
  source 1..337: mol_type = other DNA
gatgttgtgatgccccaaactccactctccctgcctgtcagtcttggagatcaagcctccatctcctgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaaatacacatgttccgctcacgttcggtgctgggaccaagctggagctgaaac

SEQ ID NO: 98 - DNA, 358 bp, synthetic construct
  misc_feature 1..358: note = 26F10chim_VH wt
  source 1..358: mol_type = other DNA
gaggttcagctgcagcagtctgggacagagcttgtgaggtcaggggcctcagtcaagttgtcctgcacaacttctggcttcaacattaaagactactatatgcactgggtgaagcagaggcctgaacagggcctggagtggattggatggattgatcctgagaatggtgatactgagtatgccccgaagttccagggcaaggccactatgactgcagacacatcctccaacacagcctacctgcaactcagcagcctgacatctgaggacactgccgtctattactgtaatgcctatgatggttactacggtgctatggactactggggtcaaggaacctcagtcaccgtctcctcag

SEQ ID NO: 99 - DNA, 333 bp, synthetic construct
  misc_feature 1..333: note = 13F7chim_VL wt
  source 1..333: mol_type = other DNA
aatatcgtgctgactcagagccccgcaagcctggccgtgagcctgggacagcgtgccaccattagttgtagagcctccgaaagcgtggactcctacggcaacagcttcatgcactggtatcagcagaagcccggccagccccctaagctgctgatctacagggcctctaatctggagtccggcatcccagctaggttctccggaagcggctctcggaccgacttcaccctgacaatcaaccccgtggaggccgacgatgtggctacatactattgtcagcagagtaatgaggacccacccaccttcgggggcggcactaaactggaaatcaag

SEQ ID NO: 100 - DNA, 358 bp, synthetic construct
  misc_feature 1..358: note = 13F7chim_VH wt
  source 1..358: mol_type = other DNA
caggtccagctgcagcagtctggggctgagctggtgaggcctggggtctcagtgaaggtttcctgcaagggttctggctacactttcactgattatgctatgcactgggtgaaacagagtcatccaaagagtctagagtggattggagttattggtacttactatggtgatgcttactacaaccagaacttcaagggcaaggccacaatgactgtagacaaatcctccagcacagcctatatggaacttgccagactgacatctgaggattctgccatctattactgtgcaagaagggctgggacgtcagactggtttgcttactggggccaagggactctggtcactgtctctgcag

SEQ ID NO: 101 - DNA, 321 bp, synthetic construct
  misc_feature 1..321: note = 11B5chim_VL wt
  source 1..321: mol_type = other DNA
gacatccagatgactcagtctccagcctccctatctgcatctgtgggagaaactgtcaccatcacatgtcgagcaagtgagaatatttacagttatttagcatggtatcagcagaaacagggaaaatctcctcagctcctggtctataatggaatcaccttagcagaaggtgtgccatcgaggttcagtggcagtggatcaggcacacaattttctctgaagatcaacagcctgcagcctgaagattttggggattattactgtcagcatcattatggtagtcctcggacgttcggtggaggcaccacgctggaaatcaaa

SEQ ID NO: 102 - DNA, 360 bp, synthetic construct
  misc_feature 1..360: note = 11B5chim_VH wt
  source 1..360: mol_type = other DNA
gaagtgcagctggtggagtctgggggagacttagtgaagcctggagggtccctgaaactctcctgtgcagcctctggattcgctttcagtgactattacatgtattgggttcgccagactccggaaaagaggctggagtgggtcgcaaccattagtgatgatggtatttacacctactatccagacagtgtgaaggggcgattcaccatctccagagacaatgccaagaacaacctgttcctgcaaatgagcagtctgaagtctgaggacacagccatgtattactgtgcaagagggggatatagagattactatgctatggactactggggtcaaggaacctcagtcaccgtctcctca

SEQ ID NO: 103 - protein, 327 aa, synthetic construct
  REGION 1..327: note = IgG4m (S228P)_
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 104 - protein, 107 aa, synthetic construct [sequence omitted]

SEQ ID NO: 105 - DNA, 15 bp, synthetic construct
  misc_feature 1..15: note = 2H9chim_VL wt_CDR1
  source 1..15: mol_type = other DNA
tcaagtataagttcc

SEQ ID NO: 106 - skipped

SEQ ID NO: 107 - DNA, 24 bp, synthetic construct
  misc_feature 1..24: note = 2H9chim_VL wt_CDR3
  source 1..24: mol_type = other DNA
cagcagtggagtagtaacctcacg

SEQ ID NO: 108 - DNA, 24 bp, synthetic construct
  misc_feature 1..24: note = 2H9chim_VH wt_CDR1
  source 1..24: mol_type = other DNA
ggattcactttcagtagctttgga

SEQ ID NO: 109 - DNA, 24 bp, synthetic construct
  misc_feature 1..24: note = 2H9chim_VH wt_CDR2
  source 1..24: mol_type = other DNA
attagtagtggcagtactaccatc

SEQ ID NO: 110 - DNA, 36 bp, synthetic construct
  misc_feature 1..36: note = 2H9chim_VH wt_CDR3
  source 1..36: mol_type = other DNA
gcaagatggacggtagtaacaaactgctttgactac

SEQ ID NO: 111 - DNA, 18 bp, synthetic construct
  misc_feature 1..18: note = 3H8chim_VL wt_CDR1
  source 1..18: mol_type = other DNA
caggatgtgagtactgct

SEQ ID NO: 112 - skipped

SEQ ID NO: 113 - DNA, 27 bp, synthetic construct
  misc_feature 1..27: note = 3H8chim_VL wt_CDR3
  source 1..27: mol_type = other DNA
cagcaacattatagtactccgtggacg

SEQ ID NO: 114 - DNA, 24 bp, synthetic construct
  misc_feature 1..24: note = 3H8chim_VH wt_CDR1
  source 1..24: mol_type = other DNA
gggtattccttcacaaactatgga

SEQ ID NO: 115 - DNA, 24 bp, synthetic construct
  misc_feature 1..24: note = 3H8chim_VH wt_CDR2
  source 1..24: mol_type = other DNA
ataaacacctacactggagagcca

SEQ ID NO: 116 - DNA, 39 bp, synthetic construct
  misc_feature 1..39: note = 3H8chim_VH wt_CDR3
  source 1..39: mol_type = other DNA
gcaaggcagccgtatgattacgtcccctcgtttccttac

SEQ ID NO: 117 - DNA, 36 bp, synthetic construct
  misc_feature 1..36: note = 26F10chim_VL wt_CDR1
  source 1..36: mol_type = other DNA
cagagccttgtacacagtaatggaaacacctattta

SEQ ID NO: 118 - skipped

SEQ ID NO: 119 - DNA, 27 bp, synthetic construct
  misc_feature 1..27: note = 26F10chim_VL wt_CDR3
  source 1..27: mol_type = other DNA
tctcaaaatacacatgttccgctcacg

SEQ ID NO: 120 - DNA, 24 bp, synthetic construct
  misc_feature 1..24: note = 26F10chim_VH wt_CDR1
  source 1..24: mol_type = other DNA
ggcttcaacattaaagactactat

SEQ ID NO: 121 - DNA, 24 bp, synthetic construct
  misc_feature 1..24: note = 26F10chim_VH wt_CDR2
  source 1..24: mol_type = other DNA
attgatcctgagaatggtgatact

SEQ ID NO: 122 - DNA, 36 bp, synthetic construct
  misc_feature 1..36: note = 26F10chim_VH wt_CDR3
  source 1..36: mol_type = other DNA
aatgcctatgatggttactacggtgctatggactac

SEQ ID NO: 123 - DNA, 30 bp, synthetic construct
  misc_feature 1..30: note = 13F7chim_VL wt_CDR1
  source 1..30: mol_type = other DNA
gaaagcgtggactcctacggcaacagcttc

SEQ ID NO: 124 - skipped

SEQ ID NO: 125 - DNA, 30 bp, synthetic construct
  misc_feature 1..30: note = 13F7chim_VL wt_CDR3
  source 1..30: mol_type = other DNA
cagcagagtaatgaggacccacccaccttc

SEQ ID NO: 126 - DNA, 24 bp, synthetic construct
  misc_feature 1..24: note = 13F7chim_VH wt_CDR1
  source 1..24: mol_type = other DNA
ggctacactttcactgattatgct

SEQ ID NO: 127 - DNA, 24 bp, synthetic construct
  misc_feature 1..24: note = 13F7chim_VH wt_CDR2
  source 1..24: mol_type = other DNA
attggtacttactatggtgatgct

SEQ ID NO: 128 - DNA, 39 bp, synthetic construct
  misc_feature 1..39: note = 13F7chim_VH wt_CDR3
  source 1..39: mol_type = other DNA
tgtgcaagaagggctgggacgtcagactggtttgcttac

SEQ ID NO: 129 - DNA, 18 bp, synthetic construct
  misc_feature 1..18: note = 11B5chim_VL wt_CDR1
  source 1..18: mol_type = other DNA
gagaatatttacagttat

SEQ ID NO: 130 - skipped

SEQ ID NO: 131 - DNA, 27 bp, synthetic construct
  misc_feature 1..27: note = 11B5chim_VL wt_CDR3
  source 1..27: mol_type = other DNA
cagcatcattatggtagtcctcggacg

SEQ ID NO: 132 - DNA, 24 bp, synthetic construct
  misc_feature 1..24: note = 11B5chim_VH wt_CDR1
  source 1..24: mol_type = other DNA
ggattcgctttcagtgactattac

SEQ ID NO: 133 - DNA, 24 bp, synthetic construct
  misc_feature 1..24: note = 11B5chim_VH wt_CDR2
  source 1..24: mol_type = other DNA
attagtgatgatggtatttacacc

SEQ ID NO: 134 - DNA, 39 bp, synthetic construct
  misc_feature 1..39: note = 11B5chim_VH wt_CDR3
  source 1..39: mol_type = other DNA
gcaagagggggatatagagattactatgctatggactac

SEQ ID NO: 135 - DNA, 984 bp, synthetic construct
  misc_feature 1..984: note = IgG4m (S228P)
  source 1..984: mol_type = other DNA
gctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaatga

SEQ ID NO: 136 - DNA, 324 bp, synthetic construct
  misc_feature 1..324: note = CLkappa
  source 1..324: mol_type = other DNA
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

(19) United States
(12) Patent Application Publication
(10) Pub. No.: US 2025/0340632 A1
(43) Pub. Date: Nov. 6, 2025

(54) SPECIFIC ANTAGONIST ANTI-SIRPg ANTIBODIES

(71) Applicant: OSE IMMUNOTHERAPEUTICS, Nantes (FR)

(72) Inventors: Lyssia Belarif, Nantes (FR); Ariane Desselle, Nantes (FR); Caroline Mary, Sainte-Pazanne (FR); Nicolas Poirier, Grandchamps Des Fontaines (FR); Virginie Thepenier, Sainte-Pazanne (FR)

(21) Appl. No.: 18/713,672

(22) PCT Filed: Nov. 29, 2022
(86) PCT No.: PCT/EP2022/083693

§ 371 Date: May 27, 2024

(30) Foreign Application Priority Data
Nov. 29, 2021 (EP) 21306660.8

(51) Int. Cl. C07K 16/28; A61K 45/06

(52) U.S. Cl. CPC C07K 16/2803; A61K 45/06; C07K 2317/24

(57) ABSTRACT

The invention relates to the field of immunotherapy. The present invention relates to new specific anti-SIRPg antibodies and their therapeutic use.

### FIELD OF THE INVENTION

The invention relates to the field of immunotherapy. The present invention relates to new specific antagonist anti-SIRPg antibodies and their therapeutic use.

### BACKGROUND OF THE INVENTION

Signal-regulatory proteins (SIRPs) constitute a family of transmembrane glycoproteins widely expressed in the immune and central nervous system and that transduce different signals.

The prototypical member of the SIRP family is SIRP-alpha (also designated as SIRPa, SIRPa, CD172a or SHPS-1). The gene coding for human SIRPa is a polymorphic gene and several variants were described in human population. The most common protein variants are SIRPa v1 (Accession number NP_542970 and P78324) and SIRPa v2 (Accession number CAA71403). SIRPa is expressed on monocytes, most subpopulations of tissue macrophages, granulocytes, subsets of dendritic cells in lymphoid tissues, some bone marrow progenitor cells, and to varying levels on neurons, with a notably high expression in synapse-rich areas of the brain, such as the granular layer of the cerebellum and the hippocampus. SIRPa is an inhibitory receptor that binds CD47 and modulates macrophage and dendritic cell function, as well as signaling pathways induced by growth factors and cell adhesion.

Another member of the SIRP family, SIRP-beta (also designated SIRPb, SIRPβ, CD172b or SIRP beta—1—Accession number NM_001083910 or Accession Q5TFQ8), was also identified. Unlike other members of the SIRP family, SIRPb does not seem to bind to CD47, and its ligand is not known yet.

The third member of the SIRP family, SIRP-gamma (also designated as SIRPg, SIRPγ, CD172g or SIRP beta 2-Accession number NM_018556 or Accession number Q9P1W8) was later identified. SIRPg is variably expressed in many human tissues, but in particular at the surface of T cells and binds to CD47 (Piccio et al., Blood, 105:6, 2005).

Only a few anti-SIRPg antibodies have been used in the past. Kwar23 antibody, an anti-SIRPa antibody was shown to also bind to SIRPg (see WO2017/178653). Brooke et al. discloses the generation of murine antibodies OX116, 117, 118 and 119. Among these antibodies, mAb OX119 was chosen as being the more specific. However, some peripheral blood myeloid cells were stained with OX119 antibodies and it was assumed that this is the consequence of cross-reactivity.

Targeting a specific epitope on SIRPg with antagonistic monoclonal antibody without effect on SIRPa/CD47 interaction, contrary to said prior art antibodies, could be a more specific strategy in order to target only T cells sparing impact on other immune cells, thus avoiding side effects in therapeutic use.

Stefanidakis M. et al. used anti-SIRPg, LSB2.20 antibody to show that CD47 is enriched in cell-cell junctions in endothelial cell and that CD47 interacting with human T-cell SIRPg plays an important role during T-cell transmigration in vitro (Stefanidakis M. et al, Blood, 2008, 112 (4): 1280-1289). Although anti-SIRPg LSB2.20 antibody used in this study was disclosed as a specific antibody, the cross-reactivity with SIRPa and antagonist activity of this anti-SIRPg were not precisely studied in this document. The Applicant previously developed anti-human SIRPg antibodies A1, A5 and A8 that selectively bind to SIRPg (WO2020/039049). However, the production yield of these antibodies has been found to be not sufficient for clinical use.

Thus, there remains a need to develop improved anti-human SIRPg antibodies having high affinity to SIRPg, which are antagonist of the SIRPg-CD47 interaction but not antagonist of the SIRPa-CD47 interaction, in particular which do not cross-react with SIRPa, that can be produced with high production yield and thus which are appropriate for clinical uses.

### SUMMARY OF THE INVENTION

The inventors developed new anti-human SIRPg antibodies that recognize and bind specifically to SIRPg antigen with high affinity. These antibodies are potent inhibitors of the interaction between human SIRPg to human CD47 but are not antagonist of the human SIRPa-CD47 interaction, (in particular do not bind to human SIRPa) and have a strong effect on the inhibition of the interferon gamma (IFNg) secretion. Moreover, the production yield of these antibodies was found to be 100 to 10000 times higher than some of the prior art antibodies and thus appropriate for clinical uses.

The present disclosure relates to an anti-SIRPg antibody or antigen-binding fragment thereof which specifically binds to SIRPg, in particular to human SIRPg, comprising:
a) a light chain variable domain comprising a VLCDR1 of SEQ ID NO: 23, a VLCDR2 of SEQ ID NO: 24, and a VLCDR3 of SEQ ID NO: 25 and a heavy chain variable domain comprising a VHCDR1 of SEQ ID NO: 26, a VHCDR2 of SEQ ID NO: 27, and a VHCDR3 of SEQ ID NO: 28,
b) a light chain variable domain comprising a VLCDR1 of SEQ ID NO: 29, a VLCDR2 of SEQ ID NO: 30, and a VLCDR3 of SEQ ID NO: 31 and a heavy chain variable domain comprising a VHCDR1 of SEQ ID NO: 32, a VHCDR2 of SEQ ID NO: 33, and a VHCDR3 of SEQ ID NO: 34,
c) a light chain variable domain comprising a VLCDR1 of SEQ ID NO: 35, a VLCDR2 of SEQ ID NO: 36, and a VLCDR3 of SEQ ID NO: 37 and a heavy chain variable domain comprising a VHCDR1 of SEQ ID NO: 38, a VHCDR2 of SEQ ID NO: 39, and a VHCDR3 of SEQ ID NO: 40,
d) a light chain variable domain comprising a VLCDR1 of SEQ ID NO: 41, a VLCDR2 of SEQ ID NO: 42, and a VLCDR3 of SEQ ID NO: 43 and a heavy chain variable domain comprising a VHCDR1 of SEQ ID NO: 44, a VHCDR2 of SEQ ID NO: 45, and a VHCDR3 of SEQ ID NO: 46, or
e) a light chain variable domain comprising a VLCDR1 of SEQ ID NO: 47, a VLCDR2 of SEQ ID NO: 48, and a VLCDR3 of SEQ ID NO: 49 and a heavy chain variable domain comprising a VHCDR1 of SEQ ID NO: 50, a VHCDR2 of SEQ ID NO: 51, and a VHCDR3 of SEQ ID NO: 52

In a particular embodiment, the antibody or antigen binding fragment thereof specifically binds to a polypeptide comprising or consisting of, in particular consisting of SEQ ID NO:1 or 2.

According to the present disclosure, said anti-SIRPg antibody or antigen binding fragment thereof as described above inhibits the binding of human CD47 to human SIRPg. In a more particular embodiment, said anti-SIRPg antibody or antigen binding fragment thereof as described above does not inhibit the binding of human SIRPa to human CD47, and preferably does not bind to human SIRPa.

In a preferred embodiment, the anti-SIRPg antibody or antigen binding fragment thereof according to the present disclosure inhibits the IFNg secretion as compared with a negative control by T cells, preferably chronically activated T cells, in particular the inhibition of IFNg secretion is over 20%, preferably over 30%, more preferably over 40%.

In a particular embodiment, the antibody or antigen binding fragment thereof according to the present disclosure is a humanized monoclonal antibody or antigen-binding thereof comprises human IgG4 heavy chain constant region, preferably comprising or consisting of SEQ ID NO: 103 and/or human Ig kappa light constant region, preferably comprising or consisting of SEQ ID NO: 104.

In a more preferred embodiment, said anti-SIRPg antibody or antigen binding fragment thereof comprises:
a light chain variable domain comprising or consisting of an amino acid sequence consisting of SEQ ID NO: 3 and a heavy chain variable domain comprising or consisting of an amino acid sequence consisting of SEQ ID NO: 4,
a light chain variable domain comprising or consisting of an amino acid sequence consisting of SEQ ID NO: 5 and a heavy chain variable domain comprising or consisting of an amino acid sequence consisting of SEQ ID NO: 6;
a light chain variable domain comprising or consisting of an amino acid sequence consisting of SEQ ID NO: 7 and a heavy chain variable domain comprising or consisting of an amino acid sequence consisting of SEQ ID NO: 8;
a light chain variable domain comprising or consisting of an amino acid sequence consisting of SEQ ID NO: 9 and a heavy chain variable domain comprising or consisting of an amino acid sequence consisting of SEQ ID NO: 10; or
a light chain variable domain comprising or consisting of an amino acid sequence consisting of SEQ ID NO: 11 and a heavy chain variable domain comprising or consisting of an amino acid sequence consisting of SEQ ID NO: 12.

In another aspect, the present disclosure relates to an isolated nucleic acid molecule or a combination of isolated nucleic acid molecules encoding the antibody or antigen-binding fragment thereof as described above.

The present disclosure also relates to a pharmaceutical composition comprising the antibody or antigen-binding fragment thereof or the isolated nucleic acid molecule or the combination of isolated nucleic acid molecules as described above, and a pharmaceutical vehicle.

Such products are particularly suitable for their uses in the prevention and/or the treatment of several diseases, in particular diseases wherein T cells are involved (in which T cells have a deleterious effect), in particular for modulating T cells proliferation and/or activation and/or migration and/or tissues infiltration by T cells, in particular wherein acting on the proliferation and/or the activation and/or the migration of T cells and/or tissues infiltration by T cells may improve the outcome of the disease.

Thus, the present disclosure also relates to an anti-SIRPg antibody or antigen binding fragment thereof, the isolated nucleic acid molecule, the combination of isolated nucleic acid molecules or the pharmaceutical composition as described above for use in the treatment of a disease in which T cells have a deleterious effect, in particular in which the proliferation and/or the activation and/or the migration of T cells and/or tissues infiltration by T cells has a deleterious effect, preferably wherein the disease is selected among the group consisting of: an auto-immune disease, inflammatory disease, an immune-metabolic disease, a cardiovascular disease caused by a systemic inflammation, and a transplant dysfunction or rejection. In a preferred embodiment, said transplant dysfunction or rejection is graft-versus-host disease. In a more preferred embodiment, said inflammatory disease is a chronic inflammatory disease such as inflammatory bowel disease including Crohn's disease or Ulcerative disease. In another particular embodiment said disease is a chronic neuroinflammatory disease.

Finally, the present disclosure relates to a combination product comprising an anti-SIRPg antibody, antigen-binding thereof, the isolated nucleic acid molecule, the combination of isolated nucleic acid molecules or the pharmaceutical composition as described above; and a second therapeutic agent selected from the group consisting of immunotherapeutic agents, immunosuppressive agents, antibiotics, probiotics and mixtures thereof, preferably wherein said immunosuppressive agent is selected from the group consisting of Cyclosporine A, tacrolimus, mycophenolate mofetil, rapamycine, steroids, anti-TNF agents, anti-IL-23 agents. In a particular embodiment, the present disclosure relates to a combination product is for simultaneous, separate or sequential use as a medicament.

### DETAILED DESCRIPTION OF THE INVENTION

### Definitions

As used herein, the term “SIRPg” has its general meaning in the art and refers to mammal SIRPg protein, preferably human SIRPg. SIRPg is a receptor-type transmembrane glycoproteins known to be involved in the negative regulation of receptor tyrosine kinase-coupled signaling processes, encoded by the gene SIRPg (Gene ID: 55423, updated on Jul. 8, 2021). A reference sequence of the human SIRPg protein corresponds to the sequence associated to the UniProtKB Accession number Q9P1W8, updated on Jun. 2, 2021.

As used herein, the term “CD47” refers to mammal CD47 protein, preferably human CD47, a membrane protein which is involved in the increase in intracellular calcium concentration that occurs upon cell adhesion to extracellular matrix. The encoded protein is also a receptor for the C-terminal cell binding domain of thrombospondin, and it may play a role in membrane transport and signal transduction. CD47 gene (Gene ID: 961, updated on Nov. 8, 2021) encodes two isoforms, CD47 isoform X2 (NCBI reference sequence: XP_005247966.1 updated on May 16 2021) and CD47 isoform X3 (NCBI reference sequence: XP_016863025.1 updated on May 16, 2021).

The term “antibody” as used herein refers to immunoglobulin molecules and immunologically active portions of immunoglobulin molecules, i.e., molecules that contain an antigen binding site that immunospecifically binds an antigen. As such, the term antibody encompasses whole antibody molecules such as four-chain antibodies comprising 2 heavy chains and 2 light chains, such as polyclonal antibodies, monoclonal antibodies or recombinant antibodies.

In natural antibodies of rodents and primates, two heavy chains are linked to each other by disulfide bonds, and each heavy chain is linked to a light chain by a disulfide bond. There are two types of light chains, lambda (λ) and kappa (κ). There are five main heavy chain classes (or isotypes) which determine the functional activity of an antibody molecule: IgM, IgD, IgG, IgA and IgE. Each chain contains distinct sequence domains. In typical IgG antibodies, the light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR).

The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from non-hypervariable or framework regions (FR) can participate in the antibody binding site or influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDRs set from each of a heavy and a light chain V region. Framework Regions (FRs) refer to amino acid sequences interposed between CDRs. Accordingly, the variable regions of the light and heavy chains typically comprise 4 framework regions and 3 CDRs of the following sequence: FR1-CDR1-FR2-CDR2-FR3-CDR3-FR4.

The skilled person is able to determine the location of the various regions/domains of antibodies by reference to the standard definitions in this respect set forth, including a reference numbering system, a reference to the numbering system of KABAT or by application of the IMGT “collier de perle” algorithm. In this respect, for the definition of the sequences of the invention, it is noted that the delimitation of the regions/domains may vary from one reference system to another. Accordingly, the regions/domains as defined in the present invention encompass sequences showing variations in length or localization of the concerned sequences within the full-length sequence of the variable domains of the antibodies, of approximately +/−10%.

The predicted CDRs (according to the numbering system of KABAT) of anti-SIRPg antibodies according to the present disclosure, such as 2H9, 3H8, 26F10, 13F7 and 11B5 are described in Table 3 below.

The term “monoclonal antibody” as used herein refers to a preparation of antibody molecules of single specificity. A monoclonal antibody displays a single binding specificity and affinity for a particular epitope. Accordingly, the term “human monoclonal antibody” refers to an antibody displaying a single binding specificity which has variable and constant regions derived from or based on human germline immunoglobulin sequences or derived from completely synthetic sequences. The method of preparing the monoclonal antibody is not relevant for the binding specificity. In an embodiment, the antibodies of the disclosure are monoclonal antibodies.

As used herein, the term “recombinant antibody” refers to antibodies which are produced, expressed, generated or isolated by recombinant means, such as antibodies which are expressed using a recombinant expression vector transfected into a host cell; antibodies isolated from a recombinant combinatorial antibody library; antibodies isolated from an animal (e.g. a mouse) which is transgenic due to human immunoglobulin genes; or antibodies which are produced, expressed, generated or isolated in any other way in which particular immunoglobulin gene sequences (such as human immunoglobulin gene sequences) are assembled with other DNA sequences. Recombinant antibodies include, for example, chimeric and humanized antibodies.

The term “antigen-binding fragment” of an antibody (or simply “antibody fragment”), as used herein, refers to one or more fragments of an antibody that retain the ability to specifically bind to an antigen (e.g., SIRPg). It has been shown that the antigen-binding function of an antibody can be performed by fragments of a full-length antibody.

Examples of binding fragments encompassed within the term “antigen-binding fragment” of an antibody include a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., 1989 Nature 341:544-546), which consists of a VH domain, or any fusion proteins comprising such antigen-binding fragments. Furthermore, although the two domains of the Fv fragment, VL and VH, are coded for by separate genes, they can be joined, using recombinant methods, by a synthetic linker that enables them to be made as a single chain protein in which the VL and VH regions pair to form monovalent molecules (known as single chain Fv (scFv); see e.g., Bird et al., 1988 Science 242:423-426; and Huston et al., 1988 Proc. Natl. Acad. Sci. 85:5879-5883). Such single chain antibodies are also intended to be encompassed within the term “antigen-binding fragment” of an antibody. These antibody fragments are obtained using conventional techniques known to those of skill in the art, and the fragments are screened for utility in the same manner as are intact antibodies.

As used herein, a “chimeric antibody” refers to an antibody in which the sequence of the variable domain derived from the germline of a mammalian species, such as a mouse, have been grafted onto the sequence of the constant domain derived from the germline of another mammalian species, such as a human. In an embodiment, the antibodies of the disclosure are chimeric antibodies.

In an embodiment, the antibodies of the disclosure are humanized antibodies. As used herein “humanized antibody” refers to an antibody in which CDR sequences derived from the germline of another mammalian species, such as mouse, have been grafted onto human framework sequences”.

As used herein, a “modified antibody” corresponds to a molecule comprising an antibody or an antigen-binding fragment thereof, wherein said antibody or functional fragment thereof is associated with a functionally different molecule. A modified antibody of the invention may be either a fusion chimeric protein or a conjugate resulting from any suitable form of attachment including covalent attachment, grafting, chemical bonding with a chemical or biological group or with a molecule, such as a PEG polymer or another protective group or molecule suitable for protection against proteases cleavage in vivo, for improvement of stability and/or half-life of the antibody or functional fragment. With similar techniques, especially by chemical coupling or grafting, a modified antibody can be prepared with a biologically active molecule, said active molecule being for example chosen among toxins, in particular Pseudomonas exotoxin A, the A-chain of plant toxin ricin or saporin toxin, especially a therapeutic active ingredient, a vector (including especially a protein vector) suitable for targeting the antibody or functional fragment to specific cells or tissues of the human body, or it may be associated with a label or with a linker, especially when fragments of the antibody are used. PEGylation of the antibody or functional fragments thereof is a particular interesting embodiment as it improves the delivery conditions of the active substance to the host, especially for a therapeutic application. PEGylation can be site specific to prevent interference with the recognition sites of the antibodies or functional fragments and can be performed with high molecular weight PEG. PEGylation can be achieved through free cysteine residues present in the sequence of the antibody or functional fragment or through added free Cysteine residues in the amino sequence of the antibody or functional fragment. In an embodiment, the anti-SIRPg antibody or antigen-binding fragment thereof of the disclosure is modified, in particular is a modified antibody.

### Anti-SIRPg Antagonist Antibody or Antigen Binding Fragment Thereof

The antibodies or antigen binding fragments thereof of the present disclosure have the property to specifically bind with high affinity to, SIRPg, in particular human SIRPg. The antibodies or antigen binding fragment thereof prevent the interaction between human CD47 and human SIRPg and have a strong effect on the inhibition of the interferon gamma (IFNg) secretion as illustrated in the examples of the present disclosure. These antibodies or antigen binding fragment thereof do not prevent the interaction between human CD47 and human SIRPa, in particular do not specifically bind to SIRPa, preferably human SIRPa.

According to the present disclosure, the antibody or antigen-binding fragment thereof according to the present disclosure specifically binds to a SIRPg antigen.

The expressions “an antibody recognizing an antigen” and “an antibody binding to an antigen” are used interchangeably herein. As used herein, the term “recognizing” refers to the ability of an antibody or antigen-binding fragment thereof to detectably bind an epitope presented on an antigen, i.e., a SIRPg antigen, particularly the extracellular loop of the receptor, more particularly an epitope of human SIRPg consisting of or localized within the polypeptide of SEQ ID NO: 1 or 2, preferably SEQ ID NO: 2. Typically, the binding affinity may be measured by methods known in the art like but not limited to Biacore analysis, Blitz analysis, ELISA assay or Scatchard plot.

In a particular embodiment, the antibody or antigen-binding fragment according to the present disclosure specifically binds to human SIRPg antigen with a EC50 of 100 ng/ml or less, preferably between 0,1 and 100 ng/ml, more particularly between 5 ng/ml and 50 ng/ml, as may be determined by ELISA binding assay. In another embodiment, it binds to a human SIRPg antigen with a EC50 of 50 ng/ml or less, 40 ng/ml or less, 30 ng/ml or less, 20 ng/ml or less, as may be determined by ELISA binding assay, or by the method disclosed in the examples of the present invention (see the methods 1.4 detailed in Examples and results detailed in FIG. 1 of the present application).

In a particular embodiment, the antibody or antigen-binding fragment according to the present disclosure binds to human SIRPg antigen with a EC50 of 3.5 μg/ml or less, particularly 3 μg/ml or less, more particularly 2.5 μg/ml or less, 2 μg/ml or less, 1.5 μg/ml or less, 1 μg/ml or less, 0.5 μg/ml or less as may be determined by binding assay by cytofluorometry, or by the method disclosed in the examples of the present invention (see the methods 1.5 detailed in Examples and results detailed in Table 5 and FIG. 2 of the present application).

The term “EC50” and as used herein refers to the measure of the effectiveness of an antibody or antigen-binding fragment thereof (e.g., an anti-SIRPg antibody or antigen-binding fragment thereof) in eliciting a biological or biochemical function (e.g., the function or activity of SIRPg) by 50%. For example, EC50 indicates how much of an anti-SIRPg antibody or antigen-binding fragment thereof is needed to elicit the activity of SIRPg by half. That is, it is the half maximal (50%) effective concentration (EC) of an anti-SIRPg antibody or antigen-binding fragment thereof (50% EC, or EC50). EC50 represents the concentration of a drug that is required for 50% effectiveness in vitro. The EC50 can be determined by techniques known in the art, for example, by constructing a dose-response curve and examining the effect of different concentrations of the anti-SIRPg antibody or antigen-binding fragment thereof on SIRPg activity, such as anti-SIRPg binding. In particular, EC50 represents the concentration of the indicated antibody or antigen-binding fragment thereof to reach 50% of the binding in ELISA binding assay or binding assay by cytofluorometry as described in the methods 1.3 and 1.5 detailed in Examples.

The present disclosure also relates to the antibody or antigen-binding fragment thereof which specifically binds to human SIRPg with an affinity constant KD higher than 10E-8 M, as may be determined by biosensor analysis.

According to one embodiment of the present disclosure said antibody or antigen-binding fragment thereof specifically binds to SIRPg e.g., does not cross-react with (does not bind to) SIRPa and/or SIRPb, particularly SIRPa, more particularly human SIRPa.

“Selective binding” or “specifically binding” typically means that the antibody or antigen-binding fragment thereof binds more strongly to a target, such as an epitope, for which it is specific as compared to the binding to another target. The antibody or antigen-binding fragment thereof binds more strongly to a first target as compared to a second target if its affinity for the first target is higher than its affinity for the second target. Typically, an antibody or antigen-binding fragment thereof binds more strongly to a first target as compared to a second target if it binds to the first target with an EC50 as mentioned above, that is lower than the EC50, for the second target. Most specifically the agent does not bind at all to the second target to a relevant extent.

The selectivity binding of an antibody or antigen-binding fragment thereof as herein disclosed may be tested using cross-reactivity assays to other SIRP members, such as SIRPa and/or SIRPb, preferably SIRPa compared with the intended target protein (SIRPg). When such cross-reactivity cannot be detected, while giving a strong signal of the intended target at the same time and at the same antibody dilution, the antibody or antigen-binding fragment thereof is typically deemed selective (see the results detailed in Example corresponding to Table 6 and FIG. 3).

An antibody or antigen-binding fragment thereof that “does not cross-react with an antigen” or “does not bind to an antigen” is intended to refer to an antibody or antigen-binding fragment thereof that binds that antigen, such as SIRPa and/or SIRPb, preferably SIRPa with a EC50, over 1000 ng/ml, particularly over 10000 ng/ml as may be determined by ELISA binding assay, more particularly with a EC50 not determinable by standard binding assays (such as ELISA binding assay). In particular, EC50 represents the concentration of the indicated antibody or antigen-binding fragment thereof to reach 50% of the binding in ELISA binding assay as described in the methods 1.3 (see the results in FIG. 3) and 1.4 detailed in Example. In preferred embodiments, such antibodies or antigen-binding fragments thereof that do not cross-react with the antigen or that does not bind to an antigen exhibit essentially undetectable binding against said antigen such as SIRPa and/or SIRPb, preferably SIRPa in standard binding assays (as may be determined by ELISA binding assay or binding assay by cytofluorometry).

In another particular embodiment, the antibody or antigen-binding fragment thereof according to the present disclosure does not inhibit SIRPa-CD47interaction, preferably human SIRPa-human CD47 interaction. In certain embodiments, such antibodies or antigen-binding fragments thereof that do not cross-react with the antigen exhibit essentially undetectable inhibition or prevention of the binding of SIRPa to CD47 as assessed for example by competition ELISA assay, as described in the examples of the present disclosure (see the methods 1.6 detailed in Example and the results in FIG. 4).

In some embodiments, an anti-SIRPg antibody or antigen-binding fragment thereof according to the present disclosure allows less than 20%, preferably less than 10%, more preferably less than 5% of physical binding inhibition between CD47 and SIRPa at a concentration higher than 400 ng/ml, more particularly higher than 800 ng/ml, and most particularly higher than 1000 ng/ml, as may be determined by competition ELISA assay.

In a more particular embodiment, a specific anti-SIRPg antibody or antigen-binding fragment thereof according to the invention has an EC50 value for the binding of SIRPg lower than 100 ng/ml, more particularly lower than 50 ng/ml as may be determined by ELISA binding assay, and an EC50 value for the binding of SIRPa and/or SIRPb, preferably SIRPa, over 1000 ng/ml, particularly over 10000 ng/ml as may be determined by ELISA binding assay, more particularly more particularly with a EC50 value for the binding of SIRPa and/or SIRPb, preferably SIRPa, not determinable by standard binding assays (such as ELISA binding assay or binding assay by cytofluorometry).

The antibody or antigen-binding fragment thereof having specific binding for SIRPg according to the present disclosure is also typically further characterized by its ability to inhibits or prevents SIRPg-CD47 interaction, preferably human SIRPg-human CD47 interaction. The antagonist property of the antibody or antigen-binding fragment thereof, in other words its ability to inhibit or prevent the binding of SIRPg to CD47 may be assessed for example by competition ELISA assay, as described in the examples of the present disclosure (see the methods 1.6 detailed in Examples and the results in FIG. 5).

In some embodiment, an anti-SIRPg antibody or antigen-binding fragment thereof according to the present disclosure allows over 60%, more preferably over 70%, more preferably over 80%, and most preferably over 90% of physical binding inhibition between CD47 and SIRPg at a concentration lower than 1000 ng/ml, more particularly lower than 800 ng/ml, and most particularly lower than 400 ng/ml. The antagonist effect can be determined using the methods 1.6 illustrated in the examples of the present application.

The antibody or antigen binding fragment thereof according to the present disclosure is further characterized in that it inhibits T cell proliferation, T cell activation, tissues infiltration and/or migration of T cells as compared to a negative control, preferably it inhibits T cell proliferation and/or T cell activation.

An antibody or antigen-binding fragment thereof of the present disclosure having such advantageous properties can be screened among anti-SIRPg antibodies using for examples the following assays.

The proliferation of T cells can be measured by incorporation of H3-thymidine. In particular, it is considered that an antibody or antigen-binding fragment thereof does inhibit the proliferation of T-cells when the proliferation of T-cells is reduced by more than 20% compared to a negative control.

The T cell activation may be assessed by analyzing the expression of CD25 and/or CD69, for example by flow cytometry, western blot, ELISA, and the like, and/or by assessing the secretion of IFNg and/or IL2, as compared to a control known for not activating T cells. In particular, the antibody or antigen-binding fragment according to the present disclosure inhibits the secretion of interferon gamma (IFNg) cytokine by T-cells and/or natural killer cells, particularly by T cells, more particularly by chronically activated T-cells, in particular decreases or inhibits the IFNg secretion by T cells as compared with a negative control, in particular the decrease or inhibition of IFNg secretion is over 20%, preferably over 30%, more preferably over 40%, again more preferably over 50%, even more preferably over 60%. Said negative control can be a medium without said antibody or a medium with an antibody known for not activating T cells. Chronically activated T cells are advantageously T cells stimulated with anti-CD3 (OKT3) and anti-CD28 antibodies as described in methods 1.7 of the Examples. The level of secretion of IFNg may be assessed for example by any methods known by skilled in the art, for example by measuring the level of IFNg secreted by T cells (e.g. chronically activated Tcells) by immunoassay, in particular ELISA as described in the examples of the present disclosure (see method 1.7 detailed in example of the present application and the FIG. 6).

Tissues infiltration and migration of T cells may be assessed by counting the number of T cells within a particular localization within the body (e.g. a tissue, an organ, a fluid, a gland) over time in presence of an anti-SIRPg antibody or antigen binding fragment thereof according to the disclosure as compared to a negative control. Tissues infiltration of T cells may be considered positive when the number of T cells increases over the time. Migration of T cells may be considered positive when the number of T cells vary over the time. T cells may be counted according to known method, like but not limited to the use of specific T cell markers.

According to an embodiment, anti-human SIRPg antibodies or antigen-binding fragments thereof according to the present disclosure include the selected recombinant anti-SIRPg antibodies 2H9, 3H8, 26F10, 13F7 and 11B5 which are structurally characterized by their heavy chain variable domain and light chain variable domain amino acid sequences as described in Table 1 below:

TABLE 1
Variable heavy and light chain amino acid sequences of
2H9, 3H8, 26F10, 13F7 and 11B5.

| | | |
|---|---|---|
| 2H9 | VL | QIVLTQSPALMSASPGEKVTMTCSASSSISSMYWYQQKPRSSPKP |
| | (SEQ ID NO: 3) | WIYLTSNLASGVPARFSGSGSGTSYSLTISSMEAEDAATYYCQQW |
| | | SSNLTFGAGTKLELK |
| | VH | DVQLVESGGGLVQPGGSRKLSCAASGFTFSSFGMHWVRQAPEKG |
| | (SEQ ID NO : 4) | LEWVAYISSGSTTIYYADTVKGRFTISRDNPKNTLFLQMTSLRSED |
| | | TAMYFCARWTVVTNCFDYWGQGTTLTVSS |
| 3H8 | VL | DIVMTQSHKFMSTSVGDRVNITCKASQDVSTAVAWYQLKPGQSP |
| | (SEQ ID NO: 5) | KLLIYWASIRHTGVPDRFTGSGSGTDYTLTISSVQAEHLALYYCQQ |
| | | HYSTPWTFGGGTKLEIK |
| | VH | QIQLVQSGPELKKPGETVNISCMASGYSFTNYGMNWVKQAPGKG |
| | (SEQ ID NO: 6) | LKWMGWINTYTGEPTYADDFKGRFAFSLETSASTAYLQINNLKNE |
| | | DMATYFCARQPYDYVPSFPYWGQGTLVTVSA |
| 26F10 | VL | DVVMPQTPLSLPVSLGDQASISCRSSQSLVHSNGNTYLHWYLQKP |
| | (SEQ ID NO: 7) | GQSPKLLIYKVSNRFSGVPDRFSGSGSGTDFTLKISRVEAEDLGVY |
| | | FCSQNTHVPLTFGAGTKLELK |
| | VH | EVQLQQSGTELVRSGASVKLSCTTSGFNIKDYYMHWVKQRPEQG |
| | (SEQ ID NO: 8) | LEWIGWIDPENGDTEYAPKFQGKATMTADTSSNTAYLQLSSLTSE |
| | | DTAVYYCNAYDGYYGAMDYWGQGTSVTVSS |
| 13F7 | VL | NIVLTQSPASLAVSLGQRATISCRASESVDSYGNSFMHWYQQKPG |
| | (SEQ ID NO: 9) | QPPKLLIYRASNLESGIPARFSGSGSRTDFTLTINPVEADDVATYYC |
| | | QQSNEDPPTFGGGTKLEIK |
| | VH | QVQLQQSGAELVRPGVSVKVSCKGSGYTFTDYAMHWVKQSHPK |
| | (SEQ ID NO: 10) | SLEWIGVIGTYYGDAYYNQNFKGKATMTVDKSSSTAYMELARLT |
| | | SEDSAIYYCARRAGTSDWFAYWGQGTLVTVSA |
| 11B5 | VL | DIQMTQSPASLSASVGETVTITCRASENIYSYLAWYQQKQGKSPQL |
| | (SEQ ID NO: 11) | LVYNGITLAEGVPSRFSGSGSGTQFSLKINSLQPEDFGDYYCQHHY |
| | | GSPRTFGGGTTLEIK |
| | VH | EVQLVESGGDLVKPGGSLKLSCAASGFAFSDYYMYWVRQTPEKR |
| | (SEQ ID NO: 12) | LEWVATISDDGIYTYYPDSVKGRFTISRDNAKNNLFLQMSSLKSED |
| | | TAMYYCARGGYRDYYAMDYWGQGTSVTVSS |

The present disclosure relates to an anti-SIRPg antibody or antigen binding fragment thereof according to the present disclosure which comprises a light chain variable domain comprising or consisting of an amino acid sequence selected from the group consisting of SEQ ID NO: 3, SEQ ID NO: 5, SEQ ID NO: 7, SEQ ID NO: 9 and SEQ ID NO: 11 and/or a heavy chain variable domain comprising or consisting of an amino acid sequence selected from the group consisting of SEQ ID NO: 4, SEQ ID NO: 6, SEQ ID NO: 8, SEQ ID NO: and SEQ ID NO: 12.

In a particular embodiment, the antibody or antigen-binding fragment thereof according to the present disclosure comprises:
a) a light chain variable region comprising or consisting of amino acid sequence of SEQ ID NO: 3 and a heavy chain variable region comprising or consisting of amino acid sequence of SEQ ID NO: 4,
b) a light chain variable region comprising or consisting of amino acid sequence of SEQ ID NO: 5 and a heavy chain variable region comprising or consisting of amino acid sequence of SEQ ID NO: 6,
c) a light chain variable region comprising or consisting of amino acid sequence of SEQ ID NO: 7 and a heavy chain variable region comprising or consisting of amino acid sequence of SEQ ID NO: 8,
d) a light chain variable region comprising or consisting of amino acid sequence of SEQ ID NO: 9 and a heavy chain variable region comprising or consisting of amino acid sequence of SEQ ID NO: 10, or
e) a light chain variable region comprising or consisting of amino acid sequence of SEQ ID NO: 11 and a heavy chain variable region comprising or consisting of amino acid sequence of SEQ ID NO: 12.

In a particular embodiment of the disclosure, the variable regions of the antibody as described above may be associated with antibody constant regions, like IgA, IgM, IgE, IgD or IgG such as IGg1, IgG2, IgG3, IgG4. Said variable regions of the antibody is preferably associated with human IgG4 heavy chain constant region, preferably comprising or consisting of SEQ ID NO: 103 and/or human kappa light chain constant region, preferably comprising or consisting of SEQ ID NO: 104.

Full length light and heavy chains amino acid sequences of selected recombinant anti-SIRPg antibodies 2H9, 3H8, 26F10, 13F7 and 11B5 are shown in Table 2.

TABLE 2
Full length heavy and light chain amino acid sequences of recombinant
anti-SIRPg antibodies 2H9, 3H8, 26F10, 13F7 and 11B5. Amino acid
sequences of constant isotype regions, IgG4m (S288P): SEQ ID NO: 103
and CL kappa: SEQ ID NO: 104, are indicated in bold.

| | | |
|---|---|---|
| 2H9 | Light Chain | QIVLTQSPALMSASPGEKVTMTCSASSSISSMYWYQQKPRSSPKP |
| | (SEQ ID NO: 13) | WIYLTSNLASGVPARFSGSGSGTSYSLTISSMEAEDAATYYCQQW |
| | | SSNLTFGAGTKLELKRTVAAPSVFIFPPSDEQLKSGTASVVCLLN |
| | | NFYPREAKVQWKVDNALQSGNSQESVTEQDSKDSTYSLSSTLT |
| | | LSKADYEKHKVYACEVTHQGLSSPVTKSFNRGEC |
| | Heavy Chain | DVQLVESGGGLVQPGGSRKLSCAASGFTFSSFGMHWVRQAPEKG |
| | (SEQ ID NO: 14) | LEWVAYISSGSTTIYYADTVKGRFTISRDNPKNTLFLQMTSLRSED |
| | | TAMYFCARWTVVTNCFDYWGQGTTLTVSSASTKGPSVFPLAPCS |
| | | RSTSESTAALGCLVKDYFPEPVTVSWNSGALTSGVHTFPAVLQ |
| | | SSGLYSLSSVVTVPSSSLGTKTYTCNVDHKPSNTKVDKRVESKY |
| | | GPPCPPCPAPEFLGGPSVFLFPPKPKDTLMISRTPEVTCVVVDV |
| | | SQEDPEVQFNWYVDGVEVHNAKTKPREEQFNSTYRVVSVLTV |
| | | LHQDWLNGKEYKCKVSNKGLPSSIEKTISKAKGQPREPQVYT |
| | | LPPSQEEMTKNQVSLTCLVKGFYPSDIAVEWESNGQPENNYKT |
| | | TPPVLDSDGSFFLYSRLTVDKSRWQEGNVFSCSVMHEALHNH |
| | | YTQKSLSLSPGK |
| 3H8 | Light Chain | DIVMTQSHKFMSTSVGDRVNITCKASQDVSTAVAWYQLKPGQSP |
| | (SEQ ID NO: 15) | KLLIYWASIRHTGVPDRFTGSGSGTDYTLTISSVQAEHLALYYCQQ |
| | | HYSTPWTFGGGTKLEIKRTVAAPSVFIFPPSDEQLKSGTASVVCL |
| | | LNNFYPREAKVQWKVDNALQSGNSQESVTEQDSKDSTYSLSST |
| | | LTLSKADYEKHKVYACEVTHQGLSSPVTKSFNRGEC |
| | Heavy Chain | QIQLVQSGPELKKPGETVNISCMASGYSFTNYGMNWVKQAPGKG |
| | (SEQ ID NO: 16) | LKWMGWINTYTGEPTYADDFKGRFAFSLETSASTAYLQINNLKNE |
| | | DMATYFCARQPYDYVPSFPYWGQGTLVTVSAASTKGPSVFPLAP |
| | | CSRSTSESTAALGCLVKDYFPEPVTVSWNSGALTSGVHTFPAV |
| | | LQSSGLYSLSSVVTVPSSSLGTKTYTCNVDHKPSNTKVDKRVES |
| | | KYGPPCPPCPAPEFLGGPSVFLFPPKPKDTLMISRTPEVTCVVV |
| | | DVSQEDPEVQFNWYVDGVEVHNAKTKPREEQFNSTYRVVSVL |
| | | TVLHQDWLNGKEYKCKVSNKGLPSSIEKTISKAKGQPREPQV |
| | | YTLPPSQEEMTKNQVSLTCLVKGFYPSDIAVEWESNGQPENNY |
| | | KTTPPVLDSDGSFFLYSRLTVDKSRWQEGNVFSCSVMHEALHN |
| | | HYTQKSLSLSPGK |
| 26F10 | Light Chain | DVVMPQTPLSLPVSLGDQASISCRSSQSLVHSNGNTYLHWYLQKP |
| | (SEQ ID NO: 17) | GQSPKLLIYKVSNRFSGVPDRFSGSGSGTDFTLKISRVEAEDLGVY |
| | | FCSQNTHVPLTFGAGTKLELKRTVAAPSVFIFPPSDEQLKSGTAS |
| | | VVCLLNNFYPREAKVQWKVDNALQSGNSQESVTEQDSKDSTY |
| | | SLSSTLTLSKADYEKHKVYACEVTHQGLSSPVTKSFNRGEC |
| | Heavy Chain | EVQLQQSGTELVRSGASVKLSCTTSGFNIKDYYMHWVKQRPEQG |
| | (SEQ ID NO: 18) | LEWIGWIDPENGDTEYAPKFQGKATMTADTSSNTAYLQLSSLTSE |
| | | DTAVYYCNAYDGYYGAMDYWGQGTSVTVSSASTKGPSVFPLAP |
| | | CSRSTSESTAALGCLVKDYFPEPVTVSWNSGALTSGVHTFPAV |
| | | LQSSGLYSLSSVVTVPSSSLGTKTYTCNVDHKPSNTKVDKRVES |
| | | KYGPPCPPCPAPEFLGGPSVFLFPPKPKDTLMISRTPEVTCVVV |
| | | DVSQEDPEVQFNWYVDGVEVHNAKTKPREEQFNSTYRVVSVL |
| | | TVLHQDWLNGKEYKCKVSNKGLPSSIEKTISKAKGQPREPQV |
| | | YTLPPSQEEMTKNQVSLTCLVKGFYPSDIAVEWESNGQPENNY |
| | | KTTPPVLDSDGSFFLYSRLTVDKSRWQEGNVFSCSVMHEALHN |
| | | HYTQKSLSLSPGK |
| 13F7 | Light Chain | NIVLTQSPASLAVSLGQRATISCRASESVDSYGNSFMHWYQQKPG |
| | (SEQ ID NO: 19) | QPPKLLIYRASNLESGIPARFSGSGSRTDFTLTINPVEADDVATYYC |
| | | QQSNEDPPTFGGGTKLEIKRTVAAPSVFIFPPSDEQLKSGTASVV |
| | | CLLNNFYPREAKVQWKVDNALQSGNSQESVTEQDSKDSTYSLS |
| | | STLTLSKADYEKHKVYACEVTHQGLSSPVTKSFNRGEC |
| | Heavy Chain | QVQLQQSGAELVRPGVSVKVSCKGSGYTFTDYAMHWVKQSHPK |
| | (SEQ ID NO: 20) | SLEWIGVIGTYYGDAYYNQNFKGKATMTVDKSSSTAYMELARLT |
| | | SEDSAIYYCARRAGTSDWFAYWGQGTLVTVSAASTKGPSVFPLA |
| | | PCSRSTSESTAALGCLVKDYFPEPVTVSWNSGALTSGVHTFPA |
| | | VLQSSGLYSLSSVVTVPSSSLGTKTYTCNVDHKPSNTKVDKRV |
| | | ESKYGPPCPPCPAPEFLGGPSVFLFPPKPKDTLMISRTPEVTCV |
| | | VVDVSQEDPEVQFNWYVDGVEVHNAKTKPREEQFNSTYRVVS |
| | | VLTVLHQDWLNGKEYKCKVSNKGLPSSIEKTISKAKGQPREP |
| | | QVYTLPPSQEEMTKNQVSLTCLVKGFYPSDIAVEWESNGQPEN |
| | | NYKTTPPVLDSDGSFFLYSRLTVDKSRWQEGNVFSCSVMHEAL |
| | | HNHYTQKSLSLSPGK |
| 11B5 | Light Chain | DIQMTQSPASLSASVGETVTITCRASENIYSYLAWYQQKQGKSPQL |
| | (SEQ ID NO: 21) | LVYNGITLAEGVPSRFSGSGSGTQFSLKINSLQPEDFGDYYCQHHY |
| | | GSPRTFGGGTTLEIKRTVAAPSVFIFPPSDEQLKSGTASVVCLLN |
| | | NFYPREAKVQWKVDNALQSGNSQESVTEQDSKDSTYSLSSTLT |
| | | LSKADYEKHKVYACEVTHQGLSSPVTKSFNRGEC |
| | Heavy Chain | EVQLVESGGDLVKPGGSLKLSCAASGFAFSDYYMYWVRQTPEKR |
| | (SEQ ID NO: 22) | LEWVATISDDGIYTYYPDSVKGRFTISRDNAKNNLFLQMSSLKSED |
| | | TAMYYCARGGYRDYYAMDYWGQGTSVTVSSASTKGPSVFPLAP |
| | | CSRSTSESTAALGCLVKDYFPEPVTVSWNSGALTSGVHTFPAV |
| | | LQSSGLYSLSSVVTVPSSSLGTKTYTCNVDHKPSNTKVDKRVES |
| | | KYGPPCPPCPAPEFLGGPSVFLFPPKPKDTLMISRTPEVTCVVV |
| | | DVSQEDPEVQFNWYVDGVEVHNAKTKPREEQFNSTYRVVSVL |
| | | TVLHQDWLNGKEYKCKVSNKGLPSSIEKTISKAKGQPREPQV |
| | | YTLPPSQEEMTKNQVSLTCLVKGFYPSDIAVEWESNGQPENNY |
| | | KTTPPVLDSDGSFFLYSRLTVDKSRWQEGNVFSCSVMHEALHN |
| | | HYTQKSLSLSPGK |

Thus, in a particular embodiment, the antibody or antigen-binding fragment thereof according to the present disclosure comprises a light chain amino acid sequence selected from the group consisting of: SEQ ID NO: 13, 15, 17, 19 and 21 and/or heavy chain amino acid sequence selected from the group consisting of: SEQ ID NO: 14, 16, 18, 20 and 22.

In a more particular embodiment, said antibody or antigen-binding fragment thereof comprises:
a) a light chain amino acid sequence of SEQ ID NO: 13 and a heavy chain amino acid sequence of SEQ ID NO: 14,
b) a light chain amino acid sequence of SEQ ID NO: 15 and a heavy chain amino acid sequence of SEQ ID NO: 16,
c) a light chain amino acid sequence of SEQ ID NO: 17 and a heavy chain amino acid sequence of SEQ ID NO: 18,
d) a light chain amino acid sequence of SEQ ID NO: 19 and a heavy chain amino acid sequence of SEQ ID NO: 20,
e) a light chain amino acid sequence of SEQ ID NO: 21 and a heavy chain amino acid sequence of SEQ ID NO: 22,

Anti-SIRPg antibodies or antigen-binding fragments thereof with amino acid sequences having at least 90%, for example, at least 95%, 96%, 97%, 98%, or 99% identity to any one of the above defined amino acid sequences are also part of the present disclosure, typically anti-SIRPg antibodies have at least equal or higher antagonist activities for SIRPg-CD47 interaction (and/or SIRPg binding affinity) and at least equal or less non-antagonist activities for SIRPa-CD47 interaction as described above than said anti-SIRPg antibodies consisting of VH and VL, in particular of heavy chain and light chain, of any one of 2H9, 3H8, 26F10, 13F7 and 11B5 antibodies.

As used herein, the percent identity between the two sequences is a function of the number of identical positions shared by the sequences (i. e., % identity=number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described below.

The percent identity between two amino acid sequences or nucleotide sequences can be determined using the algorithm of E. Meyers and W. Miller (Comput. Appl. Biosci., 4:11-17, 1988) which has been incorporated into the ALIGN program (version 2.0), using a PAM120 weight residue table, a gap length penalty of 12 and a gap penalty of 4. Alternatively, the percent identity between two amino acid sequences or nucleotide sequences can be determined using the Needleman and Wunsch (J. Mol, Biol. 48:444-453, 1970) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6. The percent identity between two nucleotide or amino acid sequences may also be determined using for example algorithms such as the BLASTN program for nucleic acid or amino acid sequences using as defaults a word length (W) of 11, an expectation (E) of 10, M=5, N=4, and a comparison of both strands.

The constant regions may be further mutated or modified, by methods known in the art, for modifying their binding capability towards Fc receptor.

As used herein, the term “IgG Fc region” is used to define the C-terminal region of an immunoglobulin heavy chain, including native sequence Fc region and variant Fc regions.

The human IgG heavy chain Fc region is generally defined as comprising the amino acid residue from position C226 or from P230 to the carboxyl-terminus of the IgG antibody. The numbering of residues in the Fc region is that of the EU index of Kabat.

Several research to develop therapeutic antibodies had led to engineer the Fc regions to optimize antibody properties allowing the generation of molecules that are better suited to the pharmacology activity required of them. The Fc region of an antibody mediates its serum half-life and effector functions, such as complement-dependent cytotoxicity (CDC), antibody-dependent cellular cytotoxicity (ADCC) and antibody-dependent cell phagocytosis (ADCP). Several mutations located at the interface between the CH2 and CH3 domains, such as T250Q/M428L and M252Y/S254T/T256E+H433K/N434F, have been shown to increase the binding affinity to FcRn and the half-life of IgG1 in vivo. However, there is not always a direct relationship between increased FcRn binding and improved half-life. One approach to improve the efficacy of a therapeutic antibody is to increase its serum persistence, thereby allowing higher circulating levels, less frequent administration and reduced doses. Engineering Fc regions may be desired to either reduce or increase the effector function of the antibody. For antibodies that target cell-surface molecules, especially those on immune cells, abrogating effector functions is required. Conversely, for antibodies intended for oncology use, increasing effector functions may improve the therapeutic activity. The four human IgG isotypes bind the activating Fcγ receptors (FcγRI, FcγRIIa, FcγRIIIa), the inhibitory FcγRIIb receptor, and the first component of complement (C1q) with different affinities, yielding very different effector functions. Binding of IgG to the FcγRs or C1q depends on residues located in the hinge region and the CH2 domain. Two regions of the CH2 domain are critical for FcγRs and C1q binding, and have unique sequences in IgG2 and IgG4.

Other anti-SIRPg antibodies or antigen-binding fragments thereof according to the present 10 disclosure which may be used include any antibodies comprising the 6 CDRs of 2H9, 3H8, 26F10, 13F7 and 11B5 as described in the Table 3 below.

TABLE 3
CDR regions of recombinant anti-SIRPg
antibodies 2H9, 3H8, 26F10, 13F7 and 11B5.

| | | | | |
|---|---|---|---|---|
| 2H9 | Light | CDR1 | SSISS | SEQ ID NO: 23 |
| | chain | CDR2 | LTS | SEQ ID NO: 24 |
| | | CDR3 | QQWSSNLT | SEQ ID NO: 25 |
| | Heavy | CDR1 | GFTFSSFG | SEQ ID NO: 26 |
| | chain | CDR2 | ISSGSTTI | SEQ ID NO: 27 |
| | | CDR3 | ARWTVVTNCFDY | SEQ ID NO: 28 |
| 3H8 | Light | CDR1 | QDVSTA | SEQ ID NO: 29 |
| | chain | CDR2 | WAS | SEQ ID NO: 30 |
| | | CDR3 | QQHYSTPWT | SEQ ID NO: 31 |
| | Heavy | CDR1 | GYSFTNYG | SEQ ID NO: 32 |
| | chain | CDR2 | INTYTGEP | SEQ ID NO: 33 |
| | | CDR3 | ARQPYDYVPSFPY | SEQ ID NO: 34 |
| 26F10 | Light | CDR1 | QSLVHSNGNTYL | SEQ ID NO: 35 |
| | chain | CDR2 | KVS | SEQ ID NO: 36 |
| | | CDR3 | SQNTHVPLT | SEQ ID NO: 37 |
| | Heavy | CDR1 | GFNIKDYY | SEQ ID NO: 38 |
| | chain | CDR2 | IDPENGDT | SEQ ID NO: 39 |
| | | CDR3 | NAYDGYYGAMDY | SEQ ID NO: 40 |
| 13F7 | Light | CDR1 | ESVDSYGNSF | SEQ ID NO: 41 |
| | chain | CDR2 | RAS | SEQ ID NO: 42 |
| | | CDR3 | QQSNEDPPTF | SEQ ID NO: 43 |
| | Heavy | CDR1 | GYTFTDYA | SEQ ID NO: 44 |
| | chain | CDR2 | IGTYYGDA | SEQ ID NO: 45 |
| | | CDR3 | CARRAGTSDWFAY | SEQ ID NO: 46 |
| 11B5 | Light | CDR1 | ENIYSY | SEQ ID NO: 47 |
| | chain | CDR2 | NGI | SEQ ID NO: 48 |
| | | CDR3 | QHHYGSPRT | SEQ ID NO: 49 |
| | Heavy | CDR1 | GFAFSDYY | SEQ ID NO: 50 |
| | chain | CDR2 | ISDDGIYT | SEQ ID NO: 51 |
| | | CDR3 | ARGGYRDYYAMDY | SEQ ID NO: 52 |

For the ease of reading, the CDR regions of light and heavy chain variable domains are called hereafter VHCDR1, VHCDR2, VHCDR3, VLCDR1, VLCDR2, VLCDR3 respectively.

The present disclosure relates to an antibody or antigen-binding fragment thereof which binds specifically to human SIRPg, comprising:
a) a light chain variable (VL) domain comprising the three CDRs VLCDR1, VLCDR2 and VLCDR3, wherein:
   VLCDR1 is selected from the group consisting of SEQ ID NO: 23, SEQ ID NO: 29, SEQ ID NO: 35, SEQ ID NO: 41 and SEQ ID NO: 47; and
   VLCDR2 is selected from the group consisting of SEQ ID NO: 24, SEQ ID NO: 30, SEQ ID NO: 36, SEQ ID NO: 42, and SEQ ID NO: 48; and
   VLCDR3 is selected from the group consisting of SEQ ID NO: 25, SEQ ID NO: 31, SEQ ID NO: 37, SEQ ID NO: 43 and SEQ ID NO: 49; and
b) a heavy chain variable (VH) domain comprising the three CDRs VHCDR1, VHCR2 and VHCDR3, wherein:
   VHCDR1 is selected from the group consisting of SEQ ID NO: 26, SEQ ID NO: 32, SEQ ID NO: 38, SEQ ID NO: 44 and SEQ ID NO: 50;
   VHCDR2 is selected from the group consisting of: SEQ ID NO: 27, SEQ ID NO: 33, SEQ ID NO: 39, SEQ ID NO: 45 and SEQ ID NO: 51; and
   VHCDR3 is selected from the group consisting of SEQ ID NO: 28, SEQ ID NO: 34, SEQ ID NO: 40, SEQ ID NO: 46 and SEQ ID NO: 52.

In a particular embodiment, the antibody or antigen-binding fragment thereof of the present disclosure, comprises:
a) a light chain variable domain comprising a VLCDR1 of SEQ ID NO: 23, a VLCDR2 of SEQ ID NO: 24, and a VLCDR3 of SEQ ID NO: 25,
b) a light chain variable domain comprising a VLCDR1 of SEQ ID NO: 29, a VLCDR2 of SEQ ID NO: 30, and a VLCDR3 of SEQ ID NO: 31,
c) a light chain variable domain comprising a VLCDR1 of SEQ ID NO: 35, a VLCDR2 of SEQ ID NO: 36, and a VLCDR3 of SEQ ID NO: 37,
d) a light chain variable domain comprising a VLCDR1 of SEQ ID NO: 41, a VLCDR2 of SEQ ID NO: 42, and a VLCDR3 of SEQ ID NO: 43, or
e) a light chain variable domain comprising a VLCDR1 of SEQ ID NO: 47, a VLCDR2 of SEQ ID NO: 48, and a VLCDR3 of SEQ ID NO: 49.

In a more particular embodiment, the anti-SIRPg antibody or antigen binding fragment thereof of the present disclosure comprises:
a) a heavy chain variable domain comprising a VHCDR1 of SEQ ID NO: 26, a VHCDR2 of SEQ ID NO: 27, and a VHCDR3 of SEQ ID NO: 28,
b) a heavy chain variable domain comprising a VHCDR1 of SEQ ID NO: 32, a VHCDR2 of SEQ ID NO: 33, and a VHCDR3 of SEQ ID NO: 34,
c) a heavy chain variable domain comprising a VHCDR1 of SEQ ID NO: 38, a VHCDR2 of SEQ ID NO: 39, and a VHCDR3 of SEQ ID NO: 40,
d) a heavy chain variable domain comprising a VHCDR1 of SEQ ID NO: 44, a VHCDR2 of SEQ ID NO: 45, and a VHCDR3 of SEQ ID NO: 46, or
e) a heavy chain variable domain comprising a VHCDR1 of SEQ ID NO: 50, a VHCDR2 of SEQ ID NO: 51, and a VHCDR3 of SEQ ID NO: 52.

In a more particular embodiment, the anti-SIRPg antibody or antigen binding fragment thereof according to the present disclosure comprises:
a) a light chain variable domain comprising a VLCDR1 of SEQ ID NO: 23, a VLCDR2 of SEQ ID NO: 24, and a VLCDR3 of SEQ ID NO: 25 and a heavy chain variable domain comprising a VHCDR1 of SEQ ID NO: 26, a VHCDR2 of SEQ ID NO: 27, and a VHCDR3 of SEQ ID NO: 28,
b) a light chain variable domain comprising a VLCDR1 of SEQ ID NO: 29, a VLCDR2 of SEQ ID NO: 30, and a VLCDR3 of SEQ ID NO: 31 and a heavy chain variable domain comprising a VHCDR1 of SEQ ID NO: 32, a VHCDR2 of SEQ ID NO: 33, and a VHCDR3 of SEQ ID NO: 34,
c) a light chain variable domain comprising a VLCDR1 of SEQ ID NO: 35, a VLCDR2 of SEQ ID NO: 36, and a VLCDR3 of SEQ ID NO: 37 and a heavy chain variable domain comprising a VHCDR1 of SEQ ID NO: 38, a VHCDR2 of SEQ ID NO: 39, and a VHCDR3 of SEQ ID NO: 40,
d) a light chain variable domain comprising a VLCDR1 of SEQ ID NO: 41, a VLCDR2 of SEQ ID NO: 42, and a VLCDR3 of SEQ ID NO: 43 and a heavy chain variable domain comprising a VHCDR1 of SEQ ID NO: 44, a VHCDR2 of SEQ ID NO: 45, and a VHCDR3 of SEQ ID NO: 46, or
e) a light chain variable domain comprising a VLCDR1 of SEQ ID NO: 47, a VLCDR2 of SEQ ID NO: 48, and a VLCDR3 of SEQ ID NO: 49 and a heavy chain variable domain comprising a VHCDR1 of SEQ ID NO: 50, a VHCDR2 of SEQ ID NO: 51, and a VHCDR3 of SEQ ID NO: 52.

It is further contemplated that antibodies or antigen-binding fragment thereof may be further screened or optimized for their affinity binding and/or antagonist activities as above defined. In particular, it is contemplated that antibodies or antigen-binding fragment thereof may have 1, 2, 3, 4, 5, 6, or more alterations in the amino acid sequence of 1, 2, 3, 4, 5, or 6 CDRs of monoclonal antibodies provided herein. It is contemplated that the amino acid in position 1, 2, 3, 4, 5, 6, 7, 8, 9, or 10 of CDR1, CDR2, CDR3, CDR4, CDR5, or CDR6 of the VJ or VDJ region of the light or heavy variable region of antibodies may have an insertion, deletion, or substitution with a conserved or non-conserved amino acid. Such amino acids that can either be substituted or constitute the substitution are disclosed above.

In some embodiments, the amino acid differences are conservative substitutions, i.e., substitutions of one amino acid with another having similar chemical or physical properties (size, charge or polarity), which substitution generally does not adversely affect the biochemical, biophysical and/or biological properties of the antibody. In particular, the substitution does not disrupt the interaction of the antibody with the SIRPg antigen and antagonistic property. Said conservative substitution(s) are advantageously chosen within one of the following five groups: Group 1-small aliphatic, non-polar or slightly polar residues (A, S, T, P, G); Group 2-polar, negatively charged residues and their amides (D, N, E, Q); Group 3-polar, positively charged residues (H, R, K); Group 4-large aliphatic, nonpolar residues (M, L, I, V, C); and Group 5-large, aromatic residues (F, Y, W).

In a particular aspect of the disclosure, it is disclosed an antibody or antigen-binding fragment thereof as defined above by their CDR domains, wherein:
a) the variable light domain comprises the amino acid sequence of the framework FR1 selected from the group consisting of: SEQ ID NO: 53, 61, 69, 77 and 85, the amino acid sequence of the framework FR2 selected from the group consisting of: SEQ ID NO: 54, 62, 70, 78 and 86, the amino acid sequence of the framework FR3 selected from the group consisting of: SEQ ID NO: 55, 63, 71, 79 and 87 and/or the amino acid sequence of the framework FR4 selected from the group consisting of: SEQ ID NO: 56, 64, 72, 80 and 88; and/or,
b) the variable heavy domain comprises the amino acid sequence of the framework FR1 selected from the group consisting of: SEQ ID NO: 57, 65, 73, 81 and 89, the amino acid sequence of the framework FR2 selected from the group consisting of: SEQ ID NO: 58, 66, 74, 82 and 90, the amino acid sequence of the framework FR3 selected from the group consisting of: SEQ ID NO: 59, 67, 75, 83 and 91 and/or the amino acid sequence of the framework FR4 selected from the group consisting of: SEQ ID NO: 60, 68, 76, 84 and 92.

In a more particular embodiment, the present disclosure relates to an antibody or antigen-binding fragment thereof as defined above by their CDR domains, wherein:
a) the variable light domain comprises the amino acid sequence of the framework:

VLFR1 of SEQ ID NO: 53, VLFR2 of SEQ ID NO: 54,
VLFR3 of SEQ ID NO: 55, and VLFR4 of SEQ ID NO:
56,
VLFR1 of SEQ ID NO: 61, VLFR2 of SEQ ID NO: 62,
VLFR3 of SEQ ID NO: 63, and VLFR4 of SEQ ID NO:
64,
VLFR1 of SEQ ID NO: 69, VLFR2 of SEQ ID NO: 70,
VLFR3 of SEQ ID NO: 71, and VLFR4 of SEQ ID NO:
72,
VLFR1 of SEQ ID NO: 77, VLFR2 of SEQ ID NO: 78,
VLFR3 of SEQ ID NO: 79, and VLFR4 of SEQ ID NO:
80,
or
VLFR1 of SEQ ID NO: 85, VLFR2 of SEQ ID NO: 86,
VLFR3 of SEQ ID NO: 87, and VLFR4 of SEQ ID NO:
88,
and/or

b) the variable heavy domain comprises the amino acid sequence of the framework:

VHFR1 of SEQ ID NO: 57, VHFR2 of SEQ ID NO: 58,
VHFR3 of SEQ ID NO: 59, and VHFR4 of SEQ ID NO:
60,
VHFR1 of SEQ ID NO: 65, VHFR2 of SEQ ID NO: 66,
VHFR3 of SEQ ID NO: 67, and VHFR4 of SEQ ID NO:
68,
VHFR1 of SEQ ID NO: 73, VHFR2 of SEQ ID NO: 74,
VHFR3 of SEQ ID NO: 75, and VHFR4 of SEQ ID NO:
76,
VHFR1 of SEQ ID NO: 81, VHFR2 of SEQ ID NO: 82,
VHFR3 of SEQ ID NO: 83, and VHFR4 of SEQ ID NO:
84,
or
VHFR1 of SEQ ID NO: 89, VHFR2 of SEQ ID NO: 90,
VHFR3 of SEQ ID NO: 91, and VHFR4 of SEQ ID NO:
92.

In a specific embodiment, said anti-SIRPg antibody or antigen-binding fragment thereof as defined above have one or more of the following properties:
(i) it binds to human SIRPg, preferably said antibody or antigen-binding fragment thereof binds human SIRPg with a EC50 of 100 ng/ml or less, preferably between 0.1 and 100 ng/ml, more particularly between 5 ng/ml and 50 ng/ml, again more particularly with a EC50 of 40 ng/ml or less, 30 ng/ml or less, 20 ng/ml or less, as may be determined by ELISA binding assay, or by the method disclosed in the examples of the present invention (see the methods 1.4 detailed in Examples and results detailed in FIG. 1 of the present application); and/or said antibody or antigen-binding fragment thereof binds human SIRPg with a EC50 of 0.,5 μg/ml or less, particularly 3 μg/ml or less, more particularly 2.5 μg/ml or less, 2 μg/ml or less, 1.5 μg/ml or less, 1 μg/ml or less, 0.5 μg/ml or less as may be determined by binding assay by cytofluorometry, or by the method disclosed in the examples of the present invention (see the methods 1.5 detailed in Examples and results detailed in Table 5 and FIG. 2 of the present application),
(ii) it does not cross-react with other SIRP members than SIRPg, such as SIRPa and/or SIRPb, preferably SIRPa, preferably said antibody or antigen-binding thereof binds to SIRPa with a EC50 over 1000 ng/ml, particularly over 10000 ng/ml as may be determined by ELISA binding assay, more particularly with a EC50 not determinable by standard binding assays (such as ELISA binding assay); and/or said antibody or antigen-binding thereof does not inhibit SIRPa-CD47 interaction, preferably said anti-SIRPg antibody or antigen-binding fragment thereof allows less than 20%, preferably less than 10%, more preferably less than 5% of physical binding inhibition between CD47 and SIRPa at a concentration higher than 400 ng/ml, more particularly higher than 800 ng/ml, and most particularly higher than 1000 ng/ml, as may be determined by competition ELISA assay.
(iii) it inhibits the interaction between human SIRPg and human CD47, preferably it allows over 60%, more preferably over 70%, more preferably over 80%, and most preferably over 90% of physical binding inhibition between CD47 and SIRPg at a concentration lower than 1000 ng/ml, more particularly lower than 800 ng/ml, and most particularly lower than 400 ng/ml as determined using the methods illustrated in the examples of the present application (see the methods 1.6 detailed in Example and the results in FIG. 5);
(iv) it inhibits T cell proliferation and/or T cell activation, preferably it inhibits T cell activation assessed by analyzing the secretion of IFNg, as compared to a control known for not activating T cells, in particular said antibody or antigen-binding fragment thereof decreases or inhibits the IFNg secretion by T cells, preferably chronically activated T cells as compared with a negative control, in particular the decrease or inhibition of IFNg secretion is over 20%, preferably over 30%, more preferably over 40%, again more preferably over 50%, even more preferably over 60% assessing by any methods known by skilled in the art, in particular as described in the examples of the present disclosure, for example by measuring the level of IFNg secreted by T cells (e.g. chronically activated T cells) by immunoassay, in particular ELISA (see method 1.7 detailed in example of the present application and the FIG. 6).

The combination of properties recited herein (antagonist of the binding of SIRPg to CD47; specific binding to SIRPg; no antagonist of the binding of SIRPa to CD47, in particular no specific binding to SIRPa and/or SIRPb, particularly no binding to SIRPa) are due to the binding of the anti-SIRPg antibody or antigen-binding fragment thereof of the disclosure to an epitope comprising, consisting of or localized within the amino acid residues of sequence SEQ ID NO: 1 or SEQ ID NO: 2.

According to the present disclosure, the anti-SIRPg antibody or antigen-binding fragment thereof as described above binds specifically to a polypeptide localized within or consisting of: SEQ ID NO: 1 or 2, preferably SEQ ID NO: 2.

As used herein, the term “epitope” means the part of an antigen to which the antibody binds. The epitopes of protein antigens can be divided into two categories, conformational epitope and linear epitope. A conformational epitope corresponds to discontinuous sections of the antigen's amino acid sequence. A linear epitope corresponds to a continuous sequence of amino acids from the antigen.

In certain embodiments that may be combined with the previous embodiments, an antibody provided herein is an antibody fragment of the above-defined antibodies.

Antibody fragments include, but are not limited to, Fab, Fab′, Fab′-SH, F(ab′)2, Fv, Unibody, and scFv fragments, diabodies, single domain or nanobodies and other fragments. Preferably, it is a monovalent antibody, such as a Fab of scFv fragments.

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites.

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516 B1). Antibody fragments can be made by various techniques, including but not limited to proteolytic digestion of an intact antibody as well as production by recombinant host cells as described herein.

### Nucleic Acid Encoding Anti-SIRPg Antibody

Also disclosed herein are the nucleic acid molecules that encode the anti-SIRPg antibody or antigen-binding fragment of the present disclosure.

Typically, said nucleic acid is a DNA or RNA molecule, which may be included in any suitable vector, such as a plasmid, cosmid, episome, artificial chromosome, phage or a viral vector. As used herein, the terms “vector”, “cloning vector” and “expression vector” mean the vehicle by which a DNA or RNA sequence (e.g., a foreign gene) can be introduced into a host cell, to transform the host and promote expression (e.g., transcription and translation) of the introduced sequence. So, a further object of the disclosure relates to a vector comprising a nucleic acid as described herein.

Examples of isolated nucleic acid molecule or combination of isolated nucleic acid molecules are those encoding the variable light and heavy chain amino acid sequences of the anti-SIRPg antibody as disclosed in the previous section, and using the genetic code and, optionally taking into account the codon bias depending on the host cell species.

In a particular embodiment, the present disclosure relates to a first isolated nucleic acid molecule comprising or consisting of a sequence encoding the variable light chain selected from the group consisting of SEQ ID NO: 93, 95, 97, 99 and 101; and/or a second isolated nucleic acid molecule comprising or consisting of a sequence encoding the variable heavy chain selected of SEQ ID No: 94, 96, 98, 100 and 102. In a particular embodiment, the sequence encoding the variable light chain and heavy chain are included in the same nucleic acid molecule.

Typically, the present disclosure relates to a first and second nucleic acid molecules encoding the variable heavy and light chain of 2H9 antibodies respectively comprising or consisting of the sequence SEQ ID NO: 93 and SEQ ID NO: 94 respectively, a first and second nucleic acid molecules encoding the variable heavy and light chain of 3H8 antibodies respectively comprising or consisting of the sequence SEQ ID NO: 95 and SEQ ID NO: 96 respectively, a first and second nucleic acid molecule encoding the variable heavy and light chain of 26F10 antibodies respectively comprising or consisting of the sequence SEQ ID NO: 97 and SEQ ID NO: 98 respectively, a first and second nucleic acid molecules encoding the variable heavy and light chain of 13F7 antibodies respectively comprising or consisting of the sequence SEQ ID NO: 99 and SEQ ID NO: 100 respectively and a first and second nucleic acid molecules encoding the variable heavy and light chain of 11B5 antibodies respectively comprising or consisting of the sequence SEQ ID NO: 101 and SEQ ID NO: 102 respectively. In a particular embodiment, the sequence encoding the variable light chain and heavy chain are included in the same nucleic acid molecule.

In a particular embodiment of the disclosure, the first and second nucleic acid molecules encoding variable heavy and light regions of the antibody as described above may be associated with nucleic acid sequence(s) encoding heavy and light constant regions respectively, preferably comprising nucleotide sequence SEQ ID NO: 135 and 136 respectively.

In another particular embodiment, the present disclosure relates to a first nucleic acid molecule encoding a light chain variable (VL) domain comprising three nucleic acid sequences encoding the three CDRs VLCDR1, VLCDR2 and VLCDR3 of 2H9, 3H8, 26F10, 13F7 and 11B5, preferably said first nucleic acid molecule comprises a nucleic acid sequence encoding VLCDR1 selected from the group consisting of: SEQ ID NO: 105, 111, 117, 123 and 129, a nucleic acid sequence encoding VLCDR2 selected from the group consisting of: SEQ ID NO: 106, 112, 118, 124 and 130, and a nucleic acid sequence encoding VLCDR3 selected from the group consisting of: SEQ ID NO: 107, 113, 119, 125 and 131 and a second nucleic acid molecule encoding a heavy chain variable (VH) domain comprising three nucleic acid sequences encoding the three CDRs VHCDR1, VHCDR2 and VHCDR3 of 2H9, 3H8, 26F10, 13F7 and 11B5, preferably said second nucleic acid molecule comprises a nucleic acid sequence encoding VHCDR1 selected from the group consisting of: SEQ ID NO: 108, 114, 120, 126 and 132, a nucleic acid sequence encoding VHCDR2 selected from the group consisting of: SEQ ID NO: 109, 115, 121, 127 and 133, and a nucleic acid sequence encoding VHCDR3 selected from the group consisting of: SEQ ID NO: 110, 116, 122, 128 and 134. In a particular embodiment, the sequence encoding the variable light chain and heavy chain are included in the same nucleic acid molecule.

In a preferred embodiment, the present disclosure relates to:
a first nucleic acid molecule encoding a light chain variable (VL) domain comprising three nucleic acid sequences encoding the three CDRs VLCDR1, VLCDR2 and VLCDR3 of 2H9 (SEQ ID NO: 105-107, respectively) and a second nucleic acid molecule encoding a heavy chain variable (VH) domain comprising the three nucleic acid sequences encoding the three CDRs VHCDR1, VHCDR2 and VHCDR3 of 2H9 (SEQ ID NO: 108-110, respectively),
a first nucleic acid molecule encoding a light chain variable (VL) domain comprising three nucleic acid sequences encoding the three CDRs VLCDR1, VLCDR2 and VLCDR3 of 3H8 (SEQ ID NO: 111 to 113, respectively) and a second nucleic acid molecule encoding a heavy chain variable (VH) domain comprising the three nucleic acid sequences encoding the three CDRs VHCDR1, VHCDR2 and VHCDR3 of 3H8 (SEQ ID NO: 114-116 respectively),
a first nucleic acid molecule encoding a light chain variable (VL) domain comprising three nucleic acid sequences encoding the three CDRs VLCDR1, VLCDR2 and VLCDR3 of 26F10 (SEQ ID NO: 117 to 119, respectively) and a second nucleic acid molecule encoding a heavy chain variable (VH) domain comprising the three nucleic acid sequences encoding the three CDRs VHCDR1, VHCDR2 and VHCDR3 of 26F10 (SEQ ID NO: 120-122 respectively),
a first nucleic acid molecule encoding a light chain variable (VL) domain comprising three nucleic acid sequences encoding the three CDRs VLCDR1, VLCDR2 and VLCDR3 of 13F7 (SEQ ID NO: 123-125, respectively) and a second nucleic acid molecule encoding a heavy chain variable (VH) domain comprising the three nucleic acid sequences encoding the three CDRs VHCDR1, VHCDR2 and VHCDR3 of 13F7 (SEQ ID NO: 126-128 respectively), or
a first nucleic acid molecule encoding a light chain variable (VL) domain comprising three nucleic acid sequences encoding the three CDRs VLCDR1, VLCDR2 and VLCDR3 of 11B5 (SEQ ID NO: 129 to 131, respectively) and a second nucleic acid molecule encoding a heavy chain variable (VH) domain comprising the three nucleic acid sequences encoding the three CDRs VHCDR1, VHCDR2 and VHCDR3 of 11B5 (SEQ ID NO: 132-134 respectively.

In a particular embodiment, the sequence encoding the variable light chain and heavy chain are included in the same nucleic acid molecule. Nucleic acids encoding anti-SIRPg antibody of the disclosure with nucleotide sequences having at least 90%, for example, at least 95%, 96%, 97%, 98%, or 99% identity to any one of the above defined nucleotides sequences are also part of the present disclosure.

The present disclosure also pertains to nucleic acid molecules that derive from the latter sequences having been optimized for protein expression in mammalian cells, for example, CHO or HEK cell lines.

The polynucleotide, vector or cell of the disclosure are useful for the production of the protein of the invention using well-known recombinant DNA techniques. The polynucleotide according to the disclosure is prepared by the conventional methods known in the art. For example, it is produced by amplification of a nucleic sequence by PCR or RT-PCR, by screening genomic DNA libraries by hybridization with a homologous probe, or else by total or partial chemical synthesis. The recombinant vectors are constructed and introduced into host cells by the conventional recombinant DNA and genetic engineering techniques, which are known in the art.

### Host Cell

A further object of the present disclosure relates to a host cell which has been transfected, infected or transformed by a nucleic acid and/or a vector according to the invention. As used herein, the term “transformation” means the introduction of a “foreign” (i.e., extrinsic, or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA bas been “transformed”.

Said host cells may be eukaryotic cells such as mammalian cells, including simian, human, dog and rodent cells. Mammalian host cells for expressing the antibodies of the disclosure include Chinese Hamster Ovary (CHO cells) including dhfr-CHO cells (described in Urlaub and Chasin, 1980) used with a DHFR selectable marker (as described in Kaufman and Sharp, 1982), CHOK1 dhfr+ cell lines, NSO myeloma cells, COS cells and SP2 cells, for example GS CHO cell lines together with GS Xceed™ gene expression system (Lonza), HEK-293 cells (ATCC CRL-1573). In a preferred embodiment, said host cells are CHO cells, or HEK-293 cells.

### Method of Producing Antibodies

Antibodies of the present disclosure can be produced in a host cell transfectoma using, for example, a combination of recombinant DNA techniques and gene transfection methods as is well known in the art (Morrison, 1985). For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. When recombinant expression vectors encoding antibody genes are introduced into mammalian host cells, the antibodies are produced by culturing the host cells for a period of time sufficient for expression of the antibody in the host cells and, optionally, secretion of the antibody into the culture medium in which the host cells are grown. Antibodies can be recovered and purified for example from the culture medium after their secretion using standard protein purification methods (Shukla et al., 2007).

### Pharmaceutical Composition

In another aspect, the present disclosure provides a composition, e.g., a pharmaceutical composition, containing an antibody, antigen binding fragment thereof, isolated nucleic acid molecule and/or a combination of isolated nucleic acid molecules disclosed herein, for example, an antibody selected from the group consisting of 2H9, 3H8, 26F10, 13F7 and 11B5 or their antigen-binding fragments, or nucleic acid molecule(s) encoding said antibody or antigen-binding fragments formulated together with a pharmaceutically acceptable carrier.

As used herein, the term “pharmaceutically acceptable” means approved by a regulatory agency or recognized pharmacopeia such as European Pharmacopeia, for use in animals and/or humans. The term “excipient” refers to a diluent, adjuvant, carrier, or vehicle with which the therapeutic agent is administered.

Any suitable pharmaceutically acceptable carrier, diluent or excipient can be used in the preparation of a pharmaceutical composition (See e.g., Remington: The Science and Practice of Pharmacy, Alfonso R. Gennaro (Editor) Mack Publishing Company, April 1997). Pharmaceutical compositions are typically sterile and stable under the conditions of manufacture and storage. Pharmaceutical compositions may be formulated as solutions (e.g. saline, dextrose solution, or buffered solution, or other pharmaceutically acceptable sterile fluids), microemulsions, liposomes, or other ordered structure suitable to accommodate a high product concentration (e.g. microparticles or nanoparticles). The carrier may be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition.

In one embodiment, the pharmaceutical composition is a parenteral pharmaceutical composition, including a composition suitable for intravenous, intraarterial, subcutaneous, intraperitoneal or intramuscular administration. These pharmaceutical compositions are exemplary only and do not limit the pharmaceutical compositions suitable for other parenteral and non-parenteral administration routes. The pharmaceutical compositions described herein can be packaged in single unit dosage or in multidosage forms.

Preferably, the pharmaceutical compositions contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

### Therapeutic Use

Given that antagonist anti-SIRPg antibody or antigen-binding fragment thereof can reduce or inhibit the proliferation and/or activation of T cells, they can favor an immunosuppressive environment and be useful for the treatment of any disease in which T cells have a deleterious effect, in particular of an autoimmune disease, a transplant dysfunction, or an inflammatory disease. Indeed, while the immune response is the host's normal and protective response to an injury or a disease, it can also cause undesired damages when it turns against host's cells.

In certain aspects, the disclosure provides to the therapeutic use of an antibody, antigen-binding fragment thereof, isolated nucleic acid molecule, combination of isolated nucleic acid molecules and/or a pharmaceutical composition according to any one of the preceding embodiments, preferably for treating, preventing or delaying a disease or a disorder in which T cells has deleterious effects, in particular an autoimmune disease, an inflammatory disease, such as a chronic inflammatory disease, a chronic neuroinflammatory disease, an immune-metabolic disease, a cardiovascular disease caused by a systemic inflammation or a transplant dysfunction.

A disease or a disorder in which T cells have deleterious effects may include accordingly any disease or disorder wherein the T cells proliferation and/or activation and/or migration and/or tissues infiltration by T cells has (have) deleterious effects, preferably wherein the T cell proliferation and/or activation has a deleterious effect.

The diseases that can be treated with immunosuppressive drugs and thus with the anti-SIRPg antibody or antigen-binding fragment thereof according to the present disclosure are well-known in the art and in particular a disease or a disorder in which T cells have deleterious effects can be selected from the group consisting of:
an auto-immune disease, in particular rheumatoid arthritis, type I diabetes, lupus, psoriasis,
an inflammatory disease such as a chronic inflammatory disease, in particular Inflammatory bowel diseases including Crohn disease and Ulcerative colitis; or a chronic neuroinflammatory disease, in particular multiple sclerosis, encephalomyelitis,
an immune-metabolic disease, in particular type II diabetes,
a cardiovascular disease caused by a systemic inflammation, in particular atherosclerosis, and
a transplant dysfunction or rejection, in particular Graft-versus-host disease,
a lymphoproliferative disease, in particular T cell lymphoma or post-transplant lymphoproliferative disease.

In a particular embodiment, the disease or disorder in which T cell proliferation and/or activation and/or migration and/or tissues infiltration has (have) a deleterious effect is a transplant dysfunction, in particular graft-versus-host disease, in particular T cell lymphoma or post-transplant lymphoproliferative disease.

The term “subject” or “patient” as used herein, refers to mammals. Mammalian species that can benefit from the disclosed methods of treatment include, but are not limited to, humans, non-human primates such as apes, chimpanzees, monkeys, and orangutans, domesticated animals, including dogs and cats, as well as livestock such as horses, cattle, pigs, sheep, and goats, or other mammalian species including, without limitation, mice, rats, guinea pigs, rabbits, hamsters, and the like.

As used herein, the term “treatment”, “treat” or “treating” refers to any act intended to ameliorate the health status of patients such as therapy, prevention, prophylaxis and retardation of the disease. In certain embodiments, such term refers to the amelioration or eradication of a disease or symptoms associated with a disease, such as according to the present disclosure the promotion of an immunosuppressive environment, in particular the prevention or the inhibition of T cells proliferation and/or activation and/or migration and/or tissues infiltration by T cells. In other embodiments, this term refers to minimizing the spread or worsening of the disease resulting from the administration of one or more therapeutic agents to a subject with such a disease.

In a further aspect, the disclosure relates to a method of treating, preventing or alleviating a disease or a disorder in which T cells have deleterious effects as described above, in a subject in need thereof that comprises administering to the subject a therapeutically effective amount of an antibody and/or antigen-binding fragment thereof and/or an isolated nucleic acid molecule and/or combination of isolated nucleic acid molecules and/or a pharmaceutical composition as described above.

In a further aspect, the disclosure relates to a method of treatment of a disease comprising the administration to a patient in need thereof of an effective amount of an anti-SIRPg antibody and/or antigen-binding thereof and/or an isolated nucleic acid molecule and/or combination of isolated nucleic acid molecules and/or a pharmaceutical composition according to the present disclosure, wherein said disease is selected among the group consisting of: an auto-immune disease, inflammatory disease, an immune-metabolic disease, a cardiovascular disease caused by a systemic inflammation, and a transplant dysfunction or rejection, in particular by inhibiting the proliferation and/or the activation and/or the migration of T cells and/or tissues infiltration by T cells.

In a further aspect, the invention relates to a method of treatment of a disease comprising the administration to a patient in need thereof of an effective amount of an anti-SIRPg antibody and/or antigen-binding thereof and/or an isolated nucleic acid molecule and/or combination of isolated nucleic acid molecules and/or a pharmaceutical composition according to the present disclosure wherein said disease is selected among the group consisting of: an auto-immune disease, inflammatory disease, an immune-metabolic disease, a cardiovascular disease caused by a systemic inflammation, and a transplant dysfunction or rejection and wherein said disease involves the proliferation and/or the activation and/or the migration of T cells and/or tissues infiltration by T cells.

In a further aspect, the subject has a disease selected from the group consisting of: an auto-immune disease, inflammatory disease, an immune-metabolic disease, a cardiovascular disease caused by a systemic inflammation, and a transplant dysfunction or rejection, involving the proliferation and/or the activation and/or the migration of T cells and/or tissues infiltration by T cells.

In the context of the invention, an “effective amount” means a therapeutically effective amount. As used herein a “therapeutically effective amount” refers to an amount effective, at dosages and for periods of time necessary to achieve the desired therapeutic result, such as prophylaxis, or treatment of a disease or a disorder in which T cells have deleterious effects. The therapeutically effective amount of the product of the invention, or pharmaceutical composition that comprises it may vary according to factors such as the disease state, age, sex, and weight of the individual, and the ability of the product or pharmaceutical composition to elicit a desired response in the individual. Dosage regimens may be adjusted to provide the optimum therapeutic response. A therapeutically effective amount is also typically one in which any toxic or detrimental effect of the product or pharmaceutical composition is outweighed by the therapeutically beneficial effects.

The product of the disclosure will be typically included in a pharmaceutical composition or medicament, optionally in combination with a pharmaceutical carrier, diluent and/or adjuvant. Such composition or medicinal product comprises the product of the disclosure in an effective amount, sufficient to provide a desired therapeutic effect, and a pharmaceutically acceptable carrier or excipient.

In one embodiment the antibody and/or antigen-binding fragment thereof and/or an isolated nucleic acid molecule and/or combination of isolated nucleic acid molecules and/or the pharmaceutical composition for its therapeutic use is administered to the subject or patient by a parenteral route, in particularly by intravenous, intraarterial, subcutaneous, intraperitoneal, or intramuscular route.

The amount of product of the disclosure that is administered to the subject or patient may vary depending on the particular circumstances of the individual subject or patient including, age, sex, and weight of the individual; the nature and stage of the disease, the aggressiveness of the disease; the route of administration; and/or concomitant medication that has been prescribed to the subject or patient. Dosage regimens may be adjusted to provide the optimum therapeutic response.

For any particular subject, specific dosage regimens may be adjusted over time according to the individual needs and the professional judgment of the person administering or supervising the administration of the compositions. Dosage ranges set forth herein are exemplary only and do not limit the dosage ranges that may be selected by medical practitioners.

The antibody or antigen-binding fragment thereof, an isolated nucleic acid molecule, the combination of isolated nucleic acid molecules and/or the pharmaceutical composition thereof may be provided at an effective dose from about 1 ng/kg body weight to about 30 mg/kg body weight, or more. In specific embodiments, the dosage may range from 1 μg/kg to about 20 mg/kg, optionally from 10 μg/kg up to 10 mg/kg or from 100 μg/kg up to 5 mg/kg (kg: subject's or patient's body weight).

In a particular embodiment of the method, the administration of an anti-SIRPg antibody, the antigen-binding thereof, the isolated nucleic acid molecule, the combination of isolated nucleic acid molecules and/or a pharmaceutical composition thereof of the disclosure decreases or inhibits the proliferation of T cells over 20% as compared with a negative control, more particularly more than 50%, and most preferably more than 70%.

In another particular embodiment the administration of an anti-SIRPg antibody, antigen-binding thereof, the isolated nucleic acid molecule, the combination of isolated nucleic acid molecules and/or the pharmaceutical composition thereof of the disclosure inhibits T-cell activation, in particular the secretion of interferon gamma (IFNg) cytokine by T-cells, in particular decreases or inhibits the IFNg secretion by T cells as compared with a negative control, in particular the decrease or inhibition of IFNg secretion is over 20%, preferably over 30%, preferably over 40%, more preferably over 50%. Said negative control can be a medium without said antibody or antibody known for not activating T cells.

The disclosure also relates to the use of a specific anti-SIRPg antibody, antigen-binding thereof, theisolated nucleic acid molecule, the combination of isolated nucleic acid molecules and/or the pharmaceutical composition according to the present disclosure in the manufacture of a medicament for the prevention and/or treatment of a disease or a disorder, in particular a human disease or a human disorder, in which T cells have a deleterious effect as described above.

In a further aspect, the invention relates to a method for inhibiting the T cells response, in particular T cells proliferation and/or T cells activation and/or the migration of T cells and/or tissues infiltration by T cells, more particularly IFNg secretion (by T cells and/or NK cells, particularly by T cells, more particularly chronically activated T cells), the method comprising:
selecting a patient having a disorder in which T cells have a deleterious effect; and
administering to the patient an effective amount of an anti-SIRPg antibody and/or antigen-binding thereof and/or an isolated nucleic acid molecule and/or combination of isolated nucleic acid molecules and/or a pharmaceutical composition according to the present disclosure.

In a further aspect, the invention relates to a method for inhibiting the T cells response, in particular T cells proliferation and/or T cells activation and/or the migration of T cells and/or tissues infiltration by T cells, more particularly IFNg secretion (by T cells and/or NK cells, particularly by T cells, more particularly chronically activated T cells), the method comprising the administration to a subject in need thereof of an effective amount of an anti-SIRPg antibody and/or antigen-binding thereof and/or an isolated nucleic acid molecule and/or combination of isolated nucleic acid molecules and/or a pharmaceutical composition according to the present disclosure.

In particular, the patient has a disease selected among the group consisting of: an auto-immune disease, inflammatory disease, an immune-metabolic disease, a cardiovascular disease caused by a systemic inflammation, and a transplant dysfunction or rejection.

In a further aspect, the patient has a disease selected from the group consisting of: an auto-immune disease, inflammatory disease, an immune-metabolic disease, a cardiovascular disease caused by a systemic inflammation, and a transplant dysfunction or rejection, involving the proliferation and/or the activation and/or the migration of T cells and/or tissues infiltration by T cells.

### Combination Product

The anti-SIRPg antibody, antigen-binding thereof, the isolated nucleic acid molecule, the combination of isolated nucleic acid molecules and/or the pharmaceutical composition as described above can be administered alone or in combination with another therapeutic agent, e.g., a second human monoclonal antibody or antigen-binding fragment thereof. For example, the anti-SIRPg antibody, antigen-binding thereof, the isolated nucleic acid molecule, the combination of isolated nucleic acid molecules and/or the pharmaceutical composition as described above is administered together with another agent, for example, an immunotherapeutic agent, an immunosuppressive agent, an erythropoiesis-stimulating agent (ESA), a pro-apoptotic agent, antibiotic, probiotic, in combination with therapeutic cell compositions, and the like.

In an embodiment, the disclosure relates to an anti-SIRPg antibody, antigen-binding thereof, the isolated nucleic acid molecule, the combination of isolated nucleic acid molecules and/or the pharmaceutical composition as described above for its use as defined above, wherein the anti-SIRPg antibody, antigen-binding thereof, the isolated nucleic acid molecule, the combination of isolated nucleic acid molecules and/or the pharmaceutical composition as described above is combined with a second therapeutic agent.

The administration of the second agent can be simultaneous or not with the administration of the specific product according to the present disclosure. Depending on the nature of the second agent, a co-administration can be prepared in the form of a combination drug, also known as a “combo”. A combo is a fixed-dose combination that includes two or more active pharmaceutical ingredients combined in a single dosage form, which is manufactured and distributed in fixed doses. But the dose regimen and/or the administration route can also differ.

In a preferred embodiment, this second therapeutic agent is selected from the group consisting of immunotherapeutic agents, immunosuppressive agents, pro-apoptotic agents, antibiotics, and probiotics.

In a preferred embodiment, this second therapeutic agent is an immunosuppressive agent selected from the group consisting of Cyclosporine A, tacrolimus, mycophenolate mofetil, rapamycine, steroids, anti-TNF agents, anti-IL-23 agents.

The present disclosure also relates to a combination product comprising:
a specific anti-SIRPg antibody, antigen-binding thereof, the isolated nucleic acid molecule, the combination of isolated nucleic acid molecules and/or the pharmaceutical composition as described above as defined above, and
a second therapeutic agent selected from the group consisting of immunotherapeutic agents, immunosuppressive agents, pro-apoptotic agents, antibiotics and probiotics, for simultaneous, separate or sequential use as a medicament, in particular for the prevention and/or the treatment of a disease or a disorder in which the activation and/or proliferation of T cells has a deleterious effect.

Dosing for such purposes may be repeated as required, e.g. daily, semi-weekly, weekly, semi-monthly, monthly, or as required during relapses.

### Kit

In another aspect, the disclosure further relates to a kit comprising antibody or antigen-binding fragment, nucleic acid, host cell or pharmaceutical composition of the invention in one or more containers. The kit may include instructions or packaging materials that describe how to administer the product contained within the kit to a patient. Containers of the kit can be of any suitable material, e.g., glass, plastic, metal, etc., and of any suitable size, shape, or configuration. In certain embodiments, the kits may include one or more ampoules or syringes that contain the products of the invention in a suitable liquid or solution form.

The invention will now be exemplified with the following examples, which are not limitative, with reference to the attached figures in which:

### FIGURE LEGENDS

FIG. 1: Binding ELISA assay on immobilized protein SIRPg-His at 1 μg/ml. Binding analysis of a) antibodies 13F7, 11B5 and 26F10 and prior art A1, A5 and Kwar23 antibodies and b) antibodies 11B5, 2H9 and 3H8 antibodies and prior art Kwar23 antibodies. Revelation was performed with a donkey anti-human antibody and revealed by colorimetry at 450 nm using TMB substrate. Isotype IgG4 m was used as a negative control.

FIG. 2: SIRPg Binding analysis of antibodies on Jurkat cells by cytofluorometry. Assessment by cytofluorometry on human Jurkat cells (previously stained with human Fc Receptor Binding Inhibitor antibody) of 13F7, 26F10, 2H9 and 3H8 and prior art LSB2.20 and OX119 antibodies binding. A) represents the percentage of positive Jurkat cells and b) the mean fluorescence intensity (MFI).

FIG. 3: SIRPa binding ELISA assay. Binding analysis of antibodies 13F7, 11B5, 2H9, 3H8 and 26F10 and prior art anti-SIRPa and Kwar23 antibodies. Revelation was performed with a donkey anti-human antibody and revealed by colorimetry at 450 nm using TMB substrate. Isotype IgG4 m was used as a negative control.

FIG. 4: Competition of antibodies with CD47 on SIRPa. Assessment by ELISA on immobilized SIRPa-His of (A) LSB2.20 and OX119 antibodies and (B) 13F7, 11B5, 26F10, 2H9 and 3H8 antibodies and prior art LSB2.20 and OX119 antibodies at different concentrations incubated with constant concentration of biotinylated CD47-Fc (3 μg/ml). Revelation was performed with streptavidin peroxidase to detect CD47 molecule and revealed by colorimetry at 450 nm using TMB substrate. Isotype IgG1m and IgG4 m were used as a negative control.

FIG. 5: Anti-SIRPg antagonist activity by competition ELISA assay with CD47. Binding analysis of (A) anti-SIRPg antibodies 13F7, 26F10, 2H9 and 3H8 and (B) 11B5, 13F7, 26F10 and 3H8 antibodies. ELISA of antibodies at different concentrations incubated with constant concentration of biotinylated CD47-Fc (3 μg/ml). Revelation was performed with streptavidin peroxidase to detect CD47 molecule and revealed by colorimetry at 450 nm or 450-630 nm using TMB substrate.

FIG. 6: Antagonist activity in biological assay. PBMC were stimulated in complete medium 3 times in plates coated with 3 μg/ml of anti-CD3 (OKT3) and 3 μg/ml of soluble anti-CD28.2. In functional assay, PBMC, exhausted or unstimulated, were activated in plates coated with 0.5 μg/ml of anti-CD3 (OKT3) and 10 μg/ml of human CD47Fc recombinant protein (#12283-H02H, SinoBiological). Then, antibodies were incubated on plates with exhausted or unstimulated PBMC for 2 days to measure IFNg secretion (#555142, BDBiosciences), and 5 days at 37° C., 5% CO2 to measure proliferation by AlamarBlue detection at 53 0nm (#DAL1100, Invitrogen). (A) percentage of IFNg secretion after 48 hours of treatment of PBMC with 1 μg/mL of 3H8 antibodies and prior art Kwar23, OX119 and LSB2.20 antibodies; (B) percentage of IFNg secretion after 48 hours of treatment of PBMC with 10 μg/mL 11B5, 26F10, 13F7 and 2H9 antibodies and prior art Kwar23 antibody.

### EXAMPLES

### 1. Methods

### 1.1 Preparation, Selection and Characterization of Mouse Anti Human SIRPg Monoclonal Antibodies

Mice were immunized with human SIRPg His (recombinant human SIRPg protein with His Tag, #11828-H08H, Sino Biological) and monoclonal antibodies were derived according to conventional techniques. The immunization protocol was performed by Diaclone SAS (Besançon, France): one microgram of human SIRPg protein was administered to 3 BALB/c strain mice in foot pad way, one day per week for the first three injections and two weeks later for the fourth injections, followed by the last injection one week after. The fifth injection at 35 days was considered as a boost before collecting ganglion cells. Hybridoma were obtained by fusing ganglion cells with the mouse myeloma X63/AG.8653. Hybridoma were first screened according to the capacity of the secreted monoclonal antibodies to bind specifically the biotinylated human SIRPg protein (#11828-H08H, Sino Biological, conjugated with biotin performed by DiaClone) and to bind specifically the human SIRPg protein at the surface of human T Lymphocytes Jurkat cell line. Hybridomas were then confirmed according to the capacity of the secreted monoclonal antibodies to bind specifically the biotinylated human SIRPg protein but not the human SIRPa His (recombinant human SIRPa protein with His Tag, #11612-H08H, Sino Biological), and to bind specifically to the Jurkat cells but not to the SIRPa positive U937 human monocyte cell line and the negative control RAJI human B Lymphoma cell line. After selection, hybridoma were cloned and cultured in RPMI complete medium. Supernatant was purified by affinity on Protein A chromatography (DiaClone, Besançon, France) with glycine 0.1M pH 2.8 elution buffer. Activity of purified antibodies were measured by ELISA against human SIRP proteins and by flow cytometry assay against SIRP cell lines and human primary blood cells.

### 1.2 Preparation of Chimeric Antibody

For construction of heavy chain of anti-SIRPgamma Ab, variable domain VH from 2H9, 3H8, 11B5, 13F7 or 26F10 sequence were synthetized and cloned by EcoRV in pcDNA3.4CHIg-hG4m expression plasmid containing Fc of human IgG4 mutated (S228P) to stabilize hinge region (pcDNA3.4 vector from Invitrogen, Toulouse). For construction of light chain of anti-SIRPgamma Ab, the identical variable domain VL from 2H9, 3H8, 11B5, 13F7 or 26F10 sequence were synthetized and cloned by BsiWI in pcDNA3.4CLIg-hk expression plasmid containing human CLkappa (pcDNA3.4 vector from Invitrogen, Toulouse). In HEK 293 Freestyle cells, the inventors have co-transfected, by lipofectamine method, plasmid containing VH2H9-FcG4 or VH3H8-FcG4 or VH11B5-FcG4 or VH13F7-FcG4 or VH26F10-FcG4 with plasmid containing VL-CLkappa. After 6 days incubation, supernatant was recovered and purified by affinity on Protein A chromatography (HiTrap, GE healthcare) with citric acid 0.1M pH3 elution buffer. Purified antibody was dialyzed in PBS and concentrated. They were quantified by UV (A280 nm) and tested in activity assay against SIRPg.

### 1.3 ELISA SIRPa Binding Assay of Chimeric Antibodies

For human SIRPa binding assay, recombinant human SIRPa (#11612-H08H, Sino Biological) was immobilized on plastic at 0.5 μg/ml (SIRPa) in carbonate buffer (pH 9.2). After saturation, purified antibodies were added in range (from initial concentration at 10 μg/ml) to measure binding. After incubation and washing, peroxidase-labeled donkey anti-human IgG (#709-035-149, Jackson Immunoresearch) was added and revealed by conventional methods.

### 1.4 ELISA Human SIRPg Binding Assay of Chimeric Antibodies

For human SIRPg binding assay, purified antibodies were captured in range (from initial concentration at 5 μg/ml) with a coated donkey anti-human IgG (H+L) (#709-005-098, Jackson Immunoresearch) immobilized at 2 μg/ml in borate buffer. After washing, biotinylated SIRPg ((#11828-H08H, Sino Biological, biotinylation performed by Ose Immunotherapeutics) was added at lug/ml and revealed by streptavidin peroxidase (#016-030-084, Jackson Immunoresearch).

### 1.5 Human SIRPg Binding Assay on Human Jurkat by Cytofluorometry

To measure binding of mouse anti-hSIRPg on human cell lines, cells were first washed in cold-PSE (Phosphate Buffer Saline with 2% of heat-inactivated bovine serum, 2 mM EDTA), blocked for 30 min at room temperature with a mix of 50-fold diluted human Fc-block (#564220, BD Pharmingen) and 20-fold diluted/heat-inactivated human serum (SAB, # H4522-100, Sigma Aldrich) and then incubated for 10 min on ice to slow the cell metabolism. Antibodies in range were incubated for 30 min on ice, and cells were washed with cold-PSE before staining for 30 min on ice with an Alexa 647-labelled goat anti-mouse IgG at 5 μg/ml (#A21236; Fisher Scientific, Illkirch, France). Samples were analyzed on CytoFlex cytofluorometer (Beckman Coulter France, Villepinte).

1.6 Human SIRPg and Human SIRPa Antagonist Activity Measured by ELISA

For antagonist activity ELISA assay, recombinant human SIRPg (#11828-H08H, Sino Biological), or recombinant human SIRPa (#11612-H08H, Sino Biological) were immobilized on plastic at respectively 2 μg/ml (SIRPg) and 0.5 μg/ml (SIRPa) in carbonate buffer (pH 9.2). During saturation, biotinylated human CD47 (#CD7-H82F6, Acrobio) were pre-incubated at a unique concentration (final concentration at 3 μg/ml) with purified antibodies in range at room temperature for 15 minutes. Pre-incubated [CD47-biot/purified antibodies] mixes were then incubated on immobilized SIRPg (O/N at room temperature) or on immobilized SIRPa (for 2 hrs at 37° C.). After incubation and washing, streptavidin-peroxidase (#016-030-084, Jackson Immunoresearch) was added and revealed by conventional methods.

### 1.7 Functional Assay

For exhausted T cells, PBMC were stimulated in complete medium 3 times in plates coated with 3 μg/ml of anti-CD3 (OKT3) and 3 μg/ml of soluble anti-CD28.2. In functional assay, PBMC, exhausted or unstimulated, were activated in plates coated with 0.5 μg/ml of anti-CD3 (OKT3) and 10 μg/ml of human CD47Fc recombinant protein (#12283-H02H, SinoBiological). Then, antibodies were incubated on plates with exhausted or unstimulated PBMC for 2 days to measure IFNg secretion (#555142, BDBiosciences), and 5 days at 37° C., 5% CO2 to measure proliferation by AlamarBlue detection at 530 nm (#DAL1100, Invitrogen).

### 2 Results

2.1 Higher productivity of 2H9, 3H8, 11B5, 26F10 and 13F7 antibodies.

The productivity of the 2H9, 3H8, 11B5, 26F10 and 13F7 antibodies is compared to prior art A1, A5 and A8 antibodies (WO2020/039049). The production yield in mammalian cell is shown in the Table 4 below:

| | Yield in |
|---|---|
| | mammalian |
| | cells (mg/L) |
| A1 | 0.001 |
| A5 | 0.012 |
| A8 | 0.015 |
| 2H9 | 6.25 |
| 3H8 | 20.50 |
| 11B5 | 19.54 |
| 26F10 | 119.00 |
| 13F7 | 22.67 |

The production yield of the 2H9, 3H8, 11B5, 26F10 and 13F7 antibodies is 100 to 100 000 times higher than prior art anti-SIRPg antibodies A1, A5 and A8, and appropriate for clinical uses.

### 2.2 Higher Binding Affinity to SIRPg of 2H9, 3H8, 11B5, 26F10 and 13F7 Antibodies

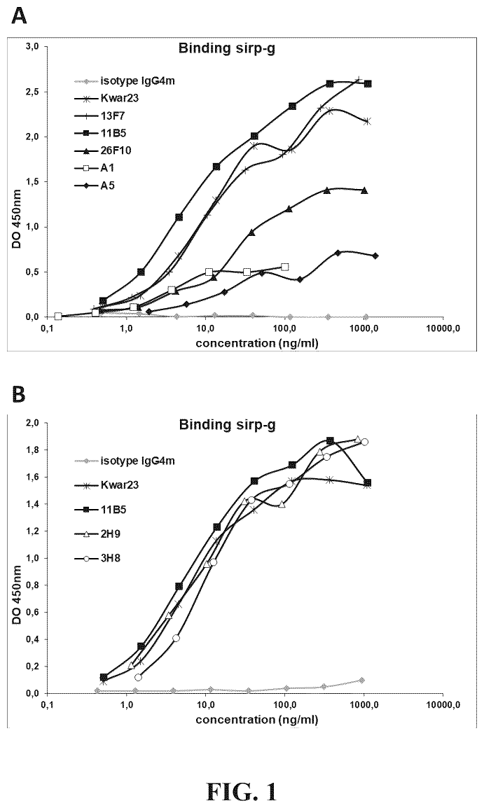

Binding affinities of purified 2H9, 3H8, 11B5, 26F10 and 13F7 antibodies measured by ELISA human SIRPg binding assay were compared to prior art antibodies Kwar23 (produced in house based on sequences disclosed in the international application WO2015/138600), A1 and A5 antibodies. As shown in FIG. 1, 2H9, 3H8, 11B5, 26F10 and 13F7 antibodies showed a similar binding affinity than the anti-SIRPalpha and gamma Kwar23 antibody and higher binding affinities to SIRPg in comparison to prior art A1 and A5 antibodies.

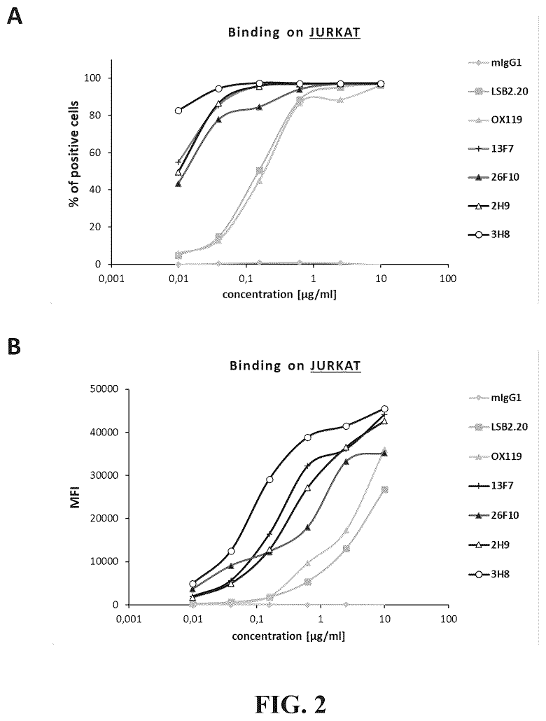

Binding affinities of purified 2H9, 3H8, 11B5, 26F10 and 13F7 antibodies on human Jurkat cells were compared to prior art LSB2.20 (Biolegend, reference 336602) and OX119 antibodies (SantaCruz reference sc-53114). As shown in FIG. 2, and in the Table 5 below, the binding affinity to SIRPg of 2H9, 3H8, 11B5, 26F10 and 13F7 measured by human SIRPg binding assay on human Jurkat cells by cytofluorometry is higher than the prior art LSB2.20 and OX119 antibodies.

TABLE 5
Human SIRPg binding assay on human Jurkat by cytofluorometry.
ED50, also named EC50 is the concentration of the indicated
antibody to reach 50% of the signal in this assay.

| | MFI |
|---|---|
| Isotype | ED50 (μg/ml) |
| IgG4m | / |
| LSB2.20 | 6.974 |
| OX119 | 3.884 |
| 13F7 | 0.281 |
| 1185 | 0.195 |
| 26F10 | 0.156 |
| 2H9 | 0.426 |
| 3H8 | 0.083 |

### 2.3 2H9, 3H8, 11B5, 26F10 and 13F7 Antibodies are Specific to SIRPg (No Binding to SIRPa—No Antagonist Activity on SIRPa-CD47 Interaction).

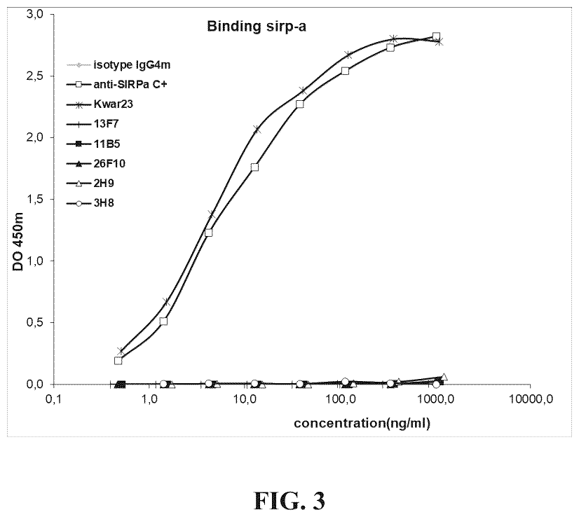

ELISA SIRPa binding assay was performed to determine the cross-reactivity of the 2H9,3H8, 11B5, 26F10 and 13F7 antibodies. As shown in FIG. 3, although Kwar23 antibody present binding affinity to SIRPa, 2H9, 3H8, 11B5, 26F10 and 13F7 antibodies do not bind to SIRPa. These results were confirmed with binding assays on U937 cells and BLITZ assay as shown in the Table 6 below:

| | | Binding | |
|---|---|---|---|
| | | to cells | |
| | SIRPa | expressing | |
| | Binding | SIRPa | |
| | (ELISA) | (U937) | Affinity |
| | (ED50- | (ED50- | to SIRPa |
| | ng/ml) | ng/ml) | (BLITZ) (KD) |
| 2H9 | / | / | / |
| 3H8 | / | / | / |
| 1185 | / | / | / |
| 13F7 | / | / | / |
| 26F10 | / | / | / |
| Kwar23 | 4.69 | 71.87 | 1.21.10⁻⁹ |
| Anti-SIRPa C+ | 5.87 | 124.44 | 2.10⁻¹⁰ |

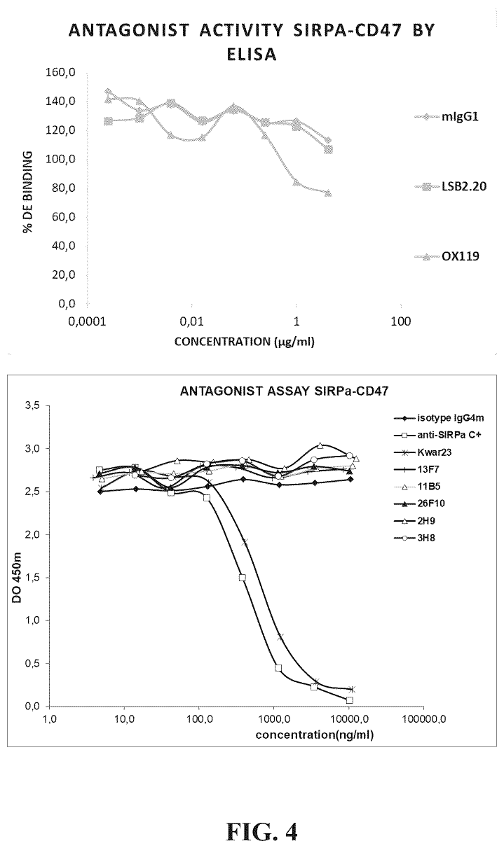

Human SIRPa antagonist activity to SIRPa-CD47 interaction were measured by ELISA. As shown in FIG. 4, although anti-SIRPalpha and gamma Kwar 23 antibody prevents the interaction of SIRPa-CD47 as well as OX119 antibody to a lesser extent; 2H9, 3H8, 11B5, 26F10 and 13F7 antibodies are unable to prevent SIRPa-CD47 interaction.

These results show that 2H9, 3H8, 11B5, 26F10 and 13F7 antibodies selectively bind to SIRPg and do not present a cross-reactivity with SIRPa. Indeed, these antibodies do not bind to human SIRPa and do not exhibit antagonist activity towards human SIRPa-CD47 interaction.

### 2.4 Efficient SIRPg Antagonist Activity on SIRPg-CD47 Interaction of 2H9, 3H8, 11B5, 26F10 and 13F7 Antibodies

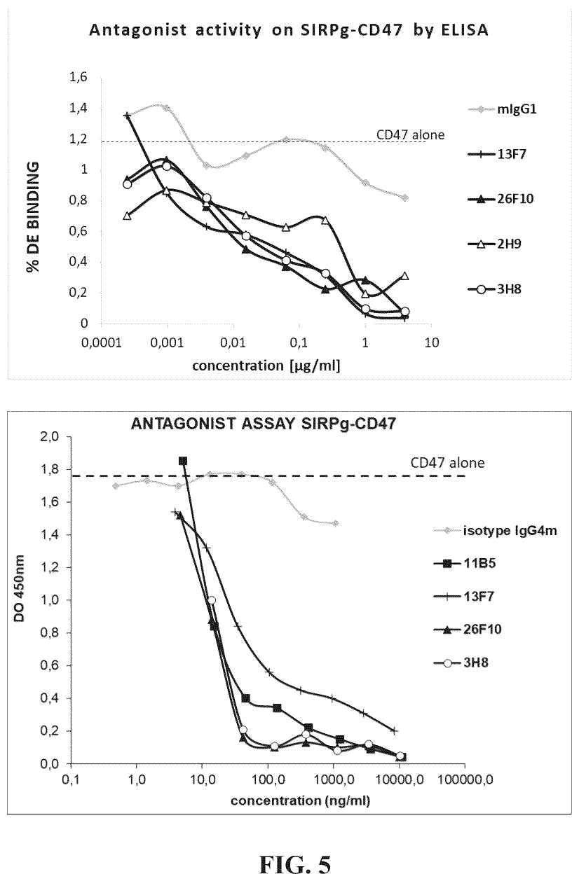

Human SIRPg antagonist activity to SIRPg-CD47 interaction of 2H9, 3H8, 11B5, 26F10 and 13F7 antibodies were measured by ELISA. As shown in FIG. 5, 2H9, 3H8, 11B5, 26F10 and 13F7 antibodies effectively prevent the interaction of SIRPg-CD47.

### 2.5 Functional Assay—Inhibition of T Cells Activation by 2H9, 3H8, 11B5, 26F10 and 13F7 Antibodies

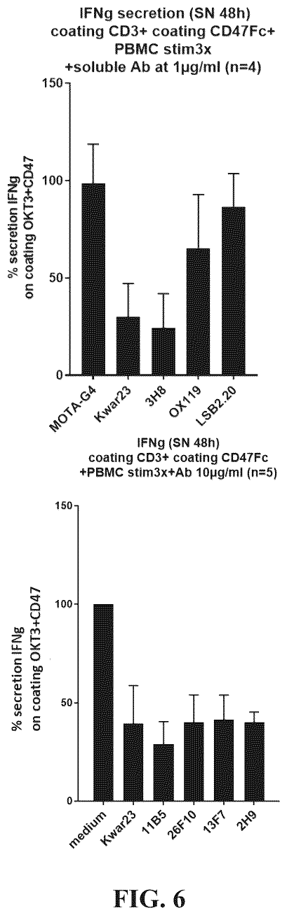

In biological assay CD47-dependent, pre-activated PBMC (CD3-CD28) were stimulated on coating CD47 and anti-CD3. The cells induced secretion of IFNg cytokine in supernatant. On this CD47-dependent biological assay, as shown in FIG. 6, the inventors observed that as the anti-SIRPalpha and gamma Kwar23 antibody (antagonist of SIRPa-CD47 interaction), the anti-SIRPg 2H9, 3H8, 11B5, 26F10 and 13F7 antibodies were able to efficiently block IFNg secretion, contrary to prior art LSB2.20 and OX119 antibodies.

In conclusion, contrary to the prior art antibodies, the antibodies of the present disclosure can be produced in high yield suitable for clinical uses, and furthermore selectively bind to SIRPg, do not cross-react with SIRPa and are not antagonist of SIRPa-CD47 interaction but are potent inhibitors of the SIRPg-CD47 interaction. Said antagonist activity on SIRPg-CD47 interaction of the antibodies of the present disclosure reduces T cell activation as shown by the decrease of IFNg secretion. Said antibodies are thus appropriate for the prevention and treatment of diseases wherein T cells have a deleterious effect.

1. An anti-SIRPg antibody or antigen binding fragment thereof which specifically binds to human SIRPg, comprising:
a) a light chain variable domain comprising a VLCDR1 of SEQ ID NO: 23, a VLCDR2 of SEQ ID NO: 24, and a VLCDR3 of SEQ ID NO: 25 and a heavy chain variable domain comprising a VHCDR1 of SEQ ID NO: 26, a VHCDR2 of SEQ ID NO: 27, and a VHCDR3 of SEQ ID NO: 28,
b) a light chain variable domain comprising a VLCDR1 of SEQ ID NO: 29, a VLCDR2 of SEQ ID NO: 30, and a VLCDR3 of SEQ ID NO: 31 and a heavy chain variable domain comprising a VHCDR1 of SEQ ID NO: 32, a VHCDR2 of SEQ ID NO: 33, and a VHCDR3 of SEQ ID NO: 34,
c) a light chain variable domain comprising a VLCDR1 of SEQ ID NO: 35, a VLCDR2 of SEQ ID NO: 36, and a VLCDR3 of SEQ ID NO: 37 and a heavy chain variable domain comprising a VHCDR1 of SEQ ID NO: 38, a VHCDR2 of SEQ ID NO: 39, and a VHCDR3 of SEQ ID NO: 40,
d) a light chain variable domain comprising a VLCDR1 of SEQ ID NO: 41, a VLCDR2 of SEQ ID NO: 42, and a VLCDR3 of SEQ ID NO: 43 and a heavy chain variable domain comprising a VHCDR1 of SEQ ID NO: 44, a VHCDR2 of SEQ ID NO: 45, and a VHCDR3 of SEQ ID NO: 46, or
e) a light chain variable domain comprising a VLCDR1 of SEQ ID NO: 47, a VLCDR2 of SEQ ID NO: 48, and a VLCDR3 of SEQ ID NO: 49 and a heavy chain variable domain comprising a VHCDR1 of SEQ ID NO: 50, a VHCDR2 of SEQ ID NO: 51, and a VHCDR3 of SEQ ID NO: 52.

2. The antibody or antigen binding fragment thereof according to claim 1, which specifically binds to a polypeptide consisting of SEQ ID NO:1 or 2.

3. The antibody or antigen binding fragment thereof according to claim 1, which inhibits the binding of human CD47 to human SIRPg.

4. The antibody or antigen binding fragment thereof according to claim 1, which does not inhibit the binding of human SIRPa to human CD47.

5. The antibody or antigen binding fragment thereof according to claim 1, which inhibits the IFNg secretion, the inhibition of IFNg secretion is over 20% as compared with a negative control.

6. The antibody or antigen binding fragment thereof according to claim 1, wherein said antibody is humanized monoclonal antibody.

7. The anti-SIRPg antibody or antigen binding fragment thereof according to claim 1 which comprises:
a light chain variable domain comprising or consisting of an amino acid sequence consisting of SEQ ID NO: 3 and a heavy chain variable domain comprising or consisting of an amino acid sequence consisting of SEQ ID NO: 4,
a light chain variable domain comprising or consisting of an amino acid sequence consisting of SEQ ID NO: 5 and a heavy chain variable domain comprising or consisting of an amino acid sequence consisting of SEQ ID NO: 6;
a light chain variable domain comprising or consisting of an amino acid sequence consisting of SEQ ID NO: 7 and a heavy chain variable domain comprising or consisting of an amino acid sequence consisting of SEQ ID NO: 8;
a light chain variable domain comprising or consisting of an amino acid sequence consisting of SEQ ID NO: 9 and a heavy chain variable domain comprising or consisting of an amino acid sequence consisting of SEQ ID NO: 10; or
a light chain variable domain comprising or consisting of an amino acid sequence consisting of SEQ ID NO: 11 and a heavy chain variable domain comprising or consisting of an amino acid sequence consisting of SEQ ID NO: 12.

8. An isolated nucleic acid molecule or a combination of isolated nucleic acid molecules encoding an antibody or antigen-binding fragment thereof according to claim 1.

9. A pharmaceutical composition comprising the antibody or antigen-binding fragment thereof according to claim 1 and a pharmaceutical vehicle.

10. (canceled)

11. (canceled)

12. (canceled)

13. (canceled)

14. (canceled)

15. A combination product comprising:
the anti-SIRPg antibody or antigen-binding thereof according to claim 1; and
a second therapeutic agent selected from the group consisting of immunotherapeutic agents, immunosuppressive agents, antibiotics, probiotics and mixtures thereof.

16. The antibody or antigen binding fragment thereof according to claim 1 which does not bind to human SIRPa.

17. The antibody or antigen binding fragment thereof according to claim 1, which inhibits the IFNg secretion by T cells as compared with a negative control without said antibody, wherein the inhibition of IFNg secretion is over 20%, as compared with a negative control.

18. The antibody or antigen binding fragment thereof according to claim 1 wherein said antibody comprises an human IgG4 heavy chain constant region or an human Ig kappa light constant region.

19. The antibody or antigen binding fragment thereof according to claim 1 wherein said antibody comprises an human IgG4 heavy chain constant region comprising or consisting of SEQ ID NO: 103 or an human Ig kappa light constant region of SEQ ID NO: 104.

20. The antibody or antigen binding fragment thereof according to claim 1 wherein said antibody comprises an human IgG4 heavy chain constant region comprising or consisting of SEQ ID NO: 103 and an human Ig kappa light constant region of SEQ ID NO: 104.

21. A method for the prevention or treatment of a disease in which T cells have a deleterious effect comprising administering a therapeutically efficient amount of an antibody or antigen binding fragment thereof according to claim 1.

22. The method of claim 21, wherein the disease is selected among the group consisting of: an auto-immune disease, inflammatory disease, an immune-metabolic disease, a cardiovascular disease caused by a systemic inflammation, and a transplant dysfunction or rejection.

23. The method of claim 22, wherein the disease is graft-versus-host disease.

24. The method of claim 21, wherein the inflammatory disease is a chronic inflammatory disease or a chronic neuroinflammatory disease.

25. The method of claim 24, wherein the chronic inflammatory disease is Crohn's disease or Ulcerative disease.
